(19) United States
(12) Patent Application Publication
(10) Pub. No.: US 2010/0330602 A1
(43) Pub. Date: Dec. 30, 2010

(54) Use of Soluble Galectin-3 (Gal-3) for Cancer Treatment

(75) Inventors: Erwin G. Van Meir, Tucker, GA (US); Fatima Khwaja, Atlanta, GA (US)

Correspondence Address: McKeon Meunier Carlin & Curfman LLC, 817 W. Peachtree Street, Suite 900, Atlanta, GA, 30308, US

(73) Assignee: Emory University, Atlanta, GA (US)

(21) Appl. No.: 12/441,978

(22) PCT Filed: Sep. 19, 2007
(86) PCT No.: PCT/US07/78857

§ 371 Date: Dec. 23, 2009

Related U.S. Application Data
(60) Provisional application No. 60/826,172, filed on Sep. 19, 2006.

(51) Int. Cl. C12Q 1/02; C12N 5/02

(52) U.S. Cl. 435/29; 435/375

(57) ABSTRACT

The present invention provides a method for preventing or treating cancer or tumorgenesis disorder comprising administering a prevention or treatment effective amount of a p53 mediated secretome component, such as Gal-3, to a patient in need thereof, thereby preventing or treating cancer or tumorgenesis disorders. Compositions and methods useful for modulating the secretome, including Gal-3, of a cell, comprising increasing extracellular levels of Gal-3, p53 expression, or expression of a downstream effector of p53, in the cell are also provided. Furthermore, methods for identifying tumor targets, diagnostic or prognostic indicators, and therapeutic strategies comprising determining extracellular levels of secreted proteins or secretomes, including Gal-3 are also provided. The present invention provides a novel tumor suppressive mechanism of p53 involving paracrine induction of apoptosis through extracellular Gal-3 levels. The invention also provides evidence that cancer cells are more susceptible to the treatment than normal cells, suggesting augmented expression of the receptor component to Gal3.

### FIELD OF THE INVENTION

The invention relates to the use of galectin-3 (Gal-3) for cancer treatment. The invention also broadly relates to a major tumor suppressor p53 and a novel tumor suppressive mechanism through extracellular secretion of proteins.

### BACKGROUND OF THE INVENTION

Traditionally, cancer formation is thought of as a cell autonomous process driven by mutations in genes that increase cell proliferation and survival, where a tumor is primarily composed of transformed cells. Increasing evidence suggests that the tumor microenvironment also contributes to the neoplasm (Hanahan and Weinberg 2000) and that the tumor-stroma interactions are an active process initiated by transforming events (Bhowmick and Moses 2005; Taieb et al. 2006).

P53 is a transcription factor that can directly control the synthesis and expression of a large number of proteins, some of which are critical effectors of its tumor suppressive activities (Harris and Levine 2005). The p53 gene is a major target for mutations in the development of many malignancies, and mutations of the p53 tumor suppressor protein play an important role in tumorigenesis through the loss of regulation of cell-cycle progression and apoptosis. The function of p53 in cell cycle control is well established and occurs through the universal transcriptional activation of the p21 cell cycle inhibitor. In contrast, the role of p53 in the induction of apoptosis is not as clearly established.

Loss or inactivation of the p53 tumor suppressor protein is seen in the majority of human cancers (Nigro et al. 1989; Hollstein et al. 1991; Levine et al. 1991; Oren 2001; Steele and Lane 2005). The widely accepted explanation for this observation is that the wt-P53 gene is undergoing negative selection under the microenvironmental growth conditions occurring during tumor development (Sidransky 1992; Ishii et al. 1999; Fulci et al. 2002). It is well established that many forms of cellular stress activate p53 with ensuing cellular outcomes such as growth arrest, senescence, or death (Wahl and Carr 2001; Schuler and Green 2005; Vousden and Prives 2005). Among the factors that activate p53 in tumor growth, hypoxia and DNA damage associated with genetic instability appear to be important contributors (Lain and Lane 2003; Hammond and Giaccia 2005). The precise p53-dependent physiological responses to these different intracellular or environmental stresses have as yet not been fully understood. Furthermore, individual components of the spectrum of p53 responses required to suppress tumorigenesis are likely to vary depending on context and organ type (Da Costa et al. 1996; Braithwaite et al. 2005)

Tumor suppressive p53 is best known for its role in maintaining genomic integrity by controlling cell cycle progression and cell survival in response to DNA damage (Steele and Lane 2005). Nevertheless, some studies have suggested that p53 can influence the tumor microenvironment through suppression of angiogenesis and tumor invasion (Miyagami and Katayama 2005; Zigrino et al 2005). These processes might be influenced by p53 through two mechanisms; the induced secretion of inhibitory factors (Van Meir et al. 1994, Dameron K M et al 1994)

or the negative regulation of secreted pro-tumorigenic proteins (Chiarugi et al. 1998; Sun et al. 2000). While p53-regulated intracellular proteins are well studied, the extracellular ones have not been systematically analyzed. Loss of tumor suppressor function during cell transformation may have cell extrinsic effects through the modulation of secreted factors. Accordingly, identification of the p53 controlled secreted proteins may clarify how p53 loss in tumors may lead to the altered regulation and response of the tumor cells to their environment.

All prior studies have focused on a cell-intrinsic mechanism of apoptosis control. A large number of pro-apoptotic mediators including Bax, Fas and PUMA are direct transcriptional targets of wt-p53 (Vogelstein et al., 2004). However, in some cell types the pro-apoptotic p53 targets are not activated by p53, leaving the question of its control over apoptosis unanswered. Recent evidence has emerged that p53 can also influence apoptosis through mechanisms independent of its transcriptional activity. P53 can trigger a cell-intrinsic cell death program by associating with Bcl2 at the mitochondrial membrane (Chipuk et al., 2004; Erster et al., 2005).

Gene therapy studies with p53 evidenced bystander therapeutic effects (Roth et al. 1996; Swisher et al. 1999; McCormick 2001). Several modes of action for the p53 bystander effect were subsequently proposed, but the precise molecular mediators were not established (Bouvet et al. 1998; Rizk et al. 1999; Seki et al. 2002). One of the prior studies proposed that in response to stress p53 may control the secretion of paracrine growth inhibitors, although their identity has remained elusive (Komarova et al. 1998).

Gal-3 is one of the beta-galactoside binding proteins that associate with carbohydrate moieties of cell surface glycoproteins or glycolipids to mediate cell-cell and cell-matrix interactions (Debray et al. 2004). Intracellular Gal-3 performs different functions based on its localization (Krzeslak and Lipinska 2004; Nakahara et al. 2005; Dumic et al. 2006). In the nucleus, it can associate with transcription factors that modulate the regulation of cell cycle-control genes to induce G1 arrest (Nakahara et al. 2005). Cytoplasmic Gal-3 can inhibit apoptosis through its interactions with the Bcl-2 protein at the mitochondrial membrane (Yang et al. 1996; Krzeslak and Lipinska 2004).

In contrast, secreted extracellular Gal-3 has been shown to signal apoptosis of human T-leukemia cell lines and human peripheral blood mononuclear cells (Fukumori et al. 2003). Since it is inhibited by lactose, this pro-apoptotic effect appears to depend on binding to cell surface glycoconjugate receptors through carbohydrate-dependent interactions. CD7 and CD29 integrin receptors may also be involved (Fukumori et al. 2003). Soluble Gal-3 interacts with over 30 lactosamine ligands on the cell surface, some of which are well characterized and include several extracellular matrix (ECM) components such as laminin, collagen IV, fibronectin vitronectin and elastin, as well as signalling receptors like CD4, CD66 and CD98, FcgRII, NCAM and Lamp-1 and 2 (Rosenberg et al. 1991; Krzeslak and Lipinska 2004). Additionally, it has been shown to bind to α1β1 and CD11b/18 integrins (Sasaki et al. 1998). However, the Gal-3 effects mediated by each of these specific receptors are heretofore unknown.

### SUMMARY OF THE INVENTION

The invention provides a method for preventing or treating cancer comprising administering a prevention or treatment effective amount of galectin or p53 mediated secretome component to a patient in need thereof, thereby preventing or treating the cancer. Preferably, the galectin used herein refers to galectin-1, galectin-3, galectin-7 (PIG3), and other galectin or galectin-like molecules or proteins that are induced by p53 and mediate cancer cell apoptosis.

The present invention further provides that p53 can exert cell-extrinsic control over tumor growth through the modulation of the secretome, including but not limited to, galectin-1, galectin-3, or other galectin secretion. In particular, the present invention provides that p53 expressing cells can induce cell death in bystander cells through p53 controlled release of galectin-3 (Gal-3), and the use of soluble Gal-3 for cancer treatment. The present invention further provides that Gal-3 secretion can be facilitated by p53 transcriptional activation of TSAP6, a key mediator of the non-traditional secretory pathway. The biological importance of p53 controlled Gal-3 secretion is demonstrated herein by showing that Gal-3 inhibits anchorage-independent tumor cell growth in vitro and strongly reduces tumor formation in vivo. Therefore, the invention provides that secretome components, such as Gal-3 itself, or as the result of the modulation of its expression by p53 or a p53 downstream effector, such as TSAP6, can be used for cancer treatment, or other diseases mediated by extracellular protein signaling.

In a preferred embodiment, the present invention provides enhanced secretion of the pro-apoptotic factor Gal-3, and use of soluble human naturally-occurred Gal-3 protein or secreted recombinant Gal-3 protein (sGal-3) based therapeutics for the treatment of cancer. The present invention further provides that the mechanism mediating p53 control over Gal-3 secretion involves transcriptional regulation of TSAP6, a transmembrane protein that plays a key role in protein secretion by exosomes. The regulation by p53 of the anti-tumor activity of extracellular Gal-3 represents a new paracrine pro-apoptotic and tumor suppressive function of p53 and provides a novel molecular mechanism for p53-mediated bystander effects in anti-cancer treatments. This bystander effect represents a novel tumor suppressive mechanism for p53 and is important for p53 gene therapy and chemo- and radiotherapies.

The present invention further provides methods of identifying p53 regulated cell secretomes, and methods for the modulation of further secreted extracellular protein, or secretome components, in a cell. The present invention provides that p53 expression, or other means of controlled administration of p53, can be used to affect the relative composition of the secretome. The present invention also provides that other downstream effectors of p53, such as TSAP6, may be used as the modulator of secreted extracellular protein. The present invention is useful for modulating the secretome of a cell, or in a patient generally, to prevent or treat diseases mediated by intercellular communication, such as tumorigenesis, angiogenesis, apoptosis, and immune-related disorders. Therefore, in certain embodiments, the present invention provides compositions and methods useful for modulating the secretome of a cell, comprising regulating p53 expression, or a downstream effector of p53, in the cell.

In preferred embodiments, the secretome modulation enhances stability and/or secretion pathways of the protein. In certain preferred embodiments, the modulation can be post-translational, including modifications affecting glycosylation, phosphorylation, or hydroxylation, which can affect the half-life of the protein or its function. In certain preferred embodiments, the modulation can relate to improving secretion, such as by increasing protein secretion through non-classical pathways, such as exocytosis, ectocytosis and transporter-mediated protein secretion. In certain preferred embodiments, the modulation can affect the regulation of tumor invasion and metastasis via induction of structural and pro-adhesion molecules used in cell-cell and cell-matrix interaction, and affect anti-migratory factors.

Therefore, in certain embodiments, the present invention provides a useful tool for screening for other effectors of the secretome which inhibit or enhance the novel regulatory systems disclosed herein. Furthermore, in certain embodiments, the present invention provides and evaluates values of cell secretomes and/or proteins identified by the present invention as diagnostic and prognostic indicators and for disease follow-up. Moreover, in certain embodiments, the present invention provides a system for development and identification of biomarkers in tumor biology, and use in clinic, as well as potential therapeutic targets for cancer therapies.

Furthermore, the present invention provides that p53 modulates interactions of the tumor with its environment. More particularly, the present invention provides the role of p53 on the tumor microenvironment through its regulation of secreted factors. In preferred embodiments, the present invention provides that extracellular levels of certain cell secretomes and/or secreted proteins are regulated (either up-regulated or down-regulated) by p53. The present invention provides that many of these proteins have known roles in cancer-related processes that are dependent on heterotypic cell-cell communication such as immune response, angiogenesis, apoptosis, extracellular matrix interaction and cell survival, and are secreted through receptor mediated classical and/or non-classical secretory pathways. However, these cell secretomes and/or secreted proteins identified by the present invention are generally not transcriptional targets of p53, indicating a novel role for p53 in the control of intracellular protein trafficking and/or secreted protein stability. Furthermore, the present invention provides that p53 affected post-translational modifications. Such modifications may alter the traffic of the protein and its function.

The present invention further provides for the use of p53 regulated secreted proteins and that p53 regulated cell secretomes and/or secreted proteins identified by the present invention can modulate interactions of tumor cells with their environment.

The present invention further provides p53 regulated secreted proteins are mediators for biological effects required for tumor growth including migration, angiogenesis, survival, cell proliferation, and immune response against neoplastic cells. In one preferred embodiment, the present invention provides several extracellular matrix (ECM) components, such as growth arrest-specific 6, collagen type XI alpha-1, proteoglycan PG-M, or proteins involved in adhesion and cell-matrix interactions, such as galectin-3, galectin-1, lysyl oxidase-like protein 2, osteopontin, alpha-catenin and beta-5 tubulin, as well as protease inhibitors, such as TIMP-3 and glioma pathogenesis-related protein, are up-regulated in the glioma cells after p53 induction. The present invention provides that the induction of these structural and pro-adhesion proteins will improve cell-cell and cell-matrix interactions, thus resulting in reduced migratory potential of tumor cells.

In addition to upregulation of anti-migratory factors, the present invention also provides other proteins directly involved in induction of migration and invasion in multiple tumor types are down-regulated by p53. These proteins include but are not limited to SPARC, MMP-2, TGF-beta, ADAM-10 and Tau. Accordingly, the present invention provides a potential new facet of p53's multimodal function as a tumor suppressor gene, through down-regulation of tumor invasion and metastasis.

In yet another preferred embodiment, the present invention provides that certain proteins involved in immune responses against tumor cells are also upregulated by p53. In one preferred embodiment, secreted osteopontin (Opn) is upregulated by p53 which one of the key cytokines for type 1 immune responses mediated by macrophages. In another preferred embodiment, immune response related proteins β-2-microglobulin (β-2M), a MHC class I molecule, and macrophage migration and myeloid leukemia inhibitory factor's secretion increased in response to p53. In yet another preferred embodiment, other immune related proteins like interleukin-8 (IL-8), attractin and ANP32A are down-regulated by p53.

In yet another preferred embodiment, the present invention provides repression of certain pro-angiogenic proteins, including but not limited to VEGF, IL-8, transforming growth factor beta, PEDF and CYR61 by p53. In yet another preferred embodiment, the present invention provides that several proteins regulating tumor proliferation and survival are regulated by p53. For instance, proteins including but not limited to brain derived neurotrophic factor (BDNF), FGF-4, RTVP1, TPM-ALK fusion oncoprotein fragment, TGF-b, PEDF, IGFBPs and granulin inhibit secretion to varying degrees in response to p53.

### BRIEF DESCRIPTION OF THE DRAWINGS

FIG. 1. Wt-p53 activated cells can kill p53-null bystander cells. Wt-p53 (green) or p53 null (red) HCT116 colon carcinoma cells were either left untreated (UT) or were treated with 25 uM etoposide (VP16), UV irradiation (60 mJ per cm²) or gamma-irradiation (10 Gy) for 24 h either alone (a,b; e,f; ij; m,n) or mixed in a 1:1 (c,g,k,o) or 3:1 ratio (d,h,l,p). Comparison with untreated cells (b) shows that P53 null cells died only when mixed with wt-p53 expressing cells (g,h; k,l; o,p) while the wt-p53 expressing cells died in response to p53-activating treatments (VP16, UV or gamma-irradiation) regardless of the presence or absence of p53 null cells (e,i,m and g,h; k,l; o,p).

FIG. 2. Gal-3 secretion is enhanced by wt-p53 and requires p53-induced TSAP6 expression. FIG. 2A. Western blot for Gal-3 levels in WCE (whole cell extracts) and CM (conditioned media) of LN-Z308 (p53 null) parental cells: 2024 (wt-p53 tet-on) and WT11 (wt-p53 tet-off) glioma cells 48 h after induction of wt-p53 expression with doxycycline (Dox; 2 μg/μl). Activation of p53 target gene p21 was used as a positive control for p53 activity and actin and thrombospondin-1 (TSP-1) were used as loading controls for WCE and CM, respectively. TSP-1 is not regulated by wt-p53 in these cells (Tenan et al. 2000).

FIG. 2B. Western blot showing extracellular accumulation (CM) of Gal-3 in response to p53 stabilization by VP16 (25 uM) in p53^{+/+} but not in p53-null HCT116 cells 18 hrs post treatment. The presence of anti-Gal-3 antibody in the media does not prevent p53 stabilization and Gal-3 secretion (lanes 5,6). Secretion of an unrelated protein (thrombospondin-1) is not affected by VP16 stabilization of p53. Intracellular levels of p53, Gal-3 in the whole cell extract (WCE) and actin are also shown.

FIG. 2C. Western blot showing extracellular accumulation (CM) of Gal-3 in response to p53 stabilization by VP16 (25 uM), UV (60 mJ per cm²) and gamma-irradiation (dose) in p53^{+/+} but not in p53-null HCT116 cells 18 hrs post treatment. The intracellular levels of Gal3, p53 and actin in the whole cell extract (WCE) are also shown. Note that the treatments stabilize p53 levels only in p53^{+/+} cells as expected. This results in increased levels of Gal3 in the CM of p53^{+/+} cells but not in p53^{-/-} cells, showing that p53 is necessary to mediate this effect.

FIG. 2D. p53 stimulates Gal-3 secretion without activating the transcription of the LGALS-3 gene. LGALS-3 is not a direct transcriptional target of wt-p53. Northern blot analysis demonstrates that Gal-3 mRNA levels are not altered by induction of wild-type (wt) or mutant (mt) p53 in doxycycline (2 ug/ml) inducible clones derived from p53 null cell line LN-Z308. Clone 2024 is tet-on for wt-p53 and 175H and 273H are tet-on for mutant p53 at the indicated codons. LNZ308-C16 (tet-on rtTA) was a control for non-specific effects of dox treatment or rtTA expression. Transcriptional activation of the p21CDKN1 gene by wt-p53 was used as a positive control.

FIG. 2E. Western blot showing endogenous wt-p53 activation in SF767 glioma cells in response to VP16 (25 uM) leads to enhanced secretion of Gal-3 (lanes 1,2). This effect is inhibited by p53 siRNA (lanes 5,6) but not control siRNA (lanes 3,4).

FIG. 2F. Northern blot analysis shows induction in TSAP6 transcript levels in response to wt-p53 expression in 2024 cells (p53 tet-on) but not in LN-Z308 (p53-null) and LN-Z308-C 16 (p53 null; rtTA) parental controls, suggesting it is not an artifact of using dox or expressing the rtTA transactivator.

FIG. 2G. Upper: Detection of TSAP6 mRNA expression by RT-PCR following UV treatment (60 mJ per cm2) for 18 hrs in p53-null (LN-Z308) and wt-p53 (SF767) glioma cells. UV treatment strongly activates wt-p53 expression in SF767 cells. TSAP6 mRNA expression was increased after UV-treatment in wt-p53 cells (lane 4) but not in p53-null cells (lane 2), confirming TSAP6 to be a target of p53. Lower: Endogenous wt-p53 activation in SF767 glioma cells in response to UV treatment leads to enhanced secretion of Gal-3. This effect is not seen in p53-null cells (LN-Z308).

FIG. 2H. TSAP6 siRNA inhibits enhanced secretion of Gal-3 in response to endogenous wt-p53 activation in SF767 cells by VP16 (25 uM).

FIG. 2I. Western blot showing extracellular accumulation (CM) of Gal-3 in response to physiological p53 stabilization by VP16 (25 uM), UV (60 mJ per cm²) and gamma-irradiation (dose?) in p53^{+/+} SF767 cells 18 hrs post treatment. The intracellular levels of Gal3, p53 and actin in the whole cell extract (WCE) are also shown. Note that all three treatments stabilize endogenous p53 levels as expected and induce endogenous Gal-3 accumulation in the conditioned media.

FIG. 23. Western blot showing endogenous wt-p53 activation in SF767 glioma cells in response to VP16 (25 uM) leads to enhanced secretion of Gal-3 (lane 1). This effect is inhibited by siRNAs for TSAP6 (lane 2), p53 (lane 3) and Fas (lane 4), but not control siRNA (lane 1).

FIG. 3: TSAP6 is necessary for Gal-3 secretion. FIG. 3A. Gal-3 secretion is enhanced in response to increased TSAP6 expression. HeLa cells were transfected with an expression vector for Gal-3 alone (lane 2), TSAP6-HA alone (lane 3) or both (lane 4). pCMV-LacZ was used as a transfection control (lane 1). The cells were switched to serum-free media 24 h post transfection and the Gal-3 levels were examined in the conditioned media (CM) and the whole cell extracts (WCE) 48 h later. Note enhanced secretion of endogenous (lane 3) and exogenous (lane 4) Gal-3 in the CM when exogenous TSAP6-HA is expressed.

FIG. 3B. siRNA-mediated inhibition of TSAP6 expression leads to reduced levels of secreted Gal-3. Human embryonic 293 cells, which have undetectable endogenous Gal-3 by Western blot, were transfected with expression vectors for Gal-3 and TSAP6-HA, with or without control or TSAP6 specific siRNAs. Cells were switched to serum free media and cell extracts and CM were analyzed 24 h later for expression of TSAP6 and Gal-3 by Western blot. Cotransfection of Gal with TSAP6-HA clearly induced the secretion of Gal-3 in the CM with a concomitant decrease in intracellular Gal-3 levels (lane 2). This was not modified by co-transfection of control siRNA (lane 3). Transfection with TSAP6-specific siRNA abrogated Gal-3 secretion (lane 4), suggesting that TSAP6 is necessary for Gal-3 secretion. Gal-3 transfection alone did not result in detectable levels of secreted Gal-3 (lane 1, 2^{nd} panel).

FIG. 4: Secreted Gal-3 induces apoptosis in tumor cells. FIG. 4A. Bright field pictures and quantification of LN229 glioma cells after 12 h treatment with either secreted Gal-3 alone or in combination with 50 μM Z-VAD general caspase inhibitor. Note inhibition of sGal-3-mediated cell death when cells are co-treated with Z-VAD. Results were quantified by crystal violet assay and experiments repeated three times in triplicate. Similar results were obtained with LN-Z308 cells (data not shown). FIG. 4B. Western blot analysis demonstrates caspase-3 (17 and 11 Kda) and PARP (85 Kda) cleavage in sGal-3 treated LN229 cells, an effect inhibited by Z-VAD. This suggests cell death through apoptosis.

FIG. 5. TSAP6 interacts and co-localizes with Gal-3. FIG. 5A. Co-immunoprecipitation demonstrates an interaction between TSAP6 and Gal-3. Immunoprecipitation with anti-Gal-3 antibody followed by western blot for HA demonstrates that Gal-3 antibody co-immunoprecipitates HA tagged TSAP6 in the HeLa cells cotransfected with TSAP6-HA and Gal-3 expression vectors (lane 5; blot 1). Similarly, anti-HA antibody is able to pull down both endogenous as well as exogenously expressed Gal-3 (lanes 4, 5; blot 3).

FIG. 5B. Co-immunofluorescence analysis shows co-localization of TSAP6 and Gal-3. HeLa cells cotransfected with Gal-3 and HA-tagged TSAP6 expression vectors were analyzed 48 h after transfection with anti-Gal-3 and anti-HA primary antibodies revealed by fluorescently labeled secondary antibodies (see materials and methods). Blue=Hoechst nuclear staining, green=HA-tagged TSAP6, Red=Gal-3 and yellow=co-localized TSAP6 and Gal-3.

FIG. 6: Secreted Gal-3 reduces tumor cell viability in vitro. FIG. 6A. Relative levels of sGal-3 found in 48 h whole cell extracts (WCE) and supernatants (CM) from 293 cells transfected with an expression vector for sGal-3 under a classical secretion signal (pUMVC7) or with a control plasmid (pCMV-LacZ). UT=untransfected. TSP-1 and actin were used as loading controls for CM and WCE, respectively.

FIG. 6B. Crystal violet assay shows reduced cell viability in the presence of CM containing sGal-3. Glioma (LN229, LN-Z308 and SF767), breast (MD468), lung (A549) and prostate (LnCaP) cancer cells were treated with CM from sGal-3 transfected (black) or pCMV-LacZ control transfected 293 cells (white) or serum-free media (gray),

FIG. 6C. Primary cultures of human endothelial cells or fibroblasts (HDMEC and HFF) do not show significant decrease in cell number in response to sGal-3 containing CM, suggesting a selective cytotoxicity of sGal-3 to tumor cells.

FIG. 6D. Purified recombinant human Gal-3 (Research Diagnostics Inc.) induces cytotoxicity in four glioma cell lines in a dose-response fashion (0, 5, 12.5, 25, 50, 100, 150 ng/ml) within 48 hrs.

FIG. 6E. CM from UV-treated wt-p53 SF767 cells induced death in a variety of cancer cells (black), while CM without wt-p53 activation (no UV treatment; white) or from UV-treated p53-null glioma cells (LN-Z308) (gray) had no effect.

FIG. 7: Conditional Gal-3 secretion reduces the tumorigenicity of malignant tumor cells in vitro and in vivo. FIG. 7A. Western blot analysis of the levels of sGal-3 expression in three LN229-L16-derived dox-inducible clones (#33, #21 and #12) selected for low, medium and high sGal-3 expression after 48 h of dox induction.

FIG. 7B. sGal-3 inhibits anchorage-independent growth. Soft-agar assays show dose-dependent inhibition of colony formation upon dox-inducible sGal-3 expression in all three clones. Dox treatment of L16 parental cells had no effect excluding non-specific effects of dox or rtTA transactivator. Left: representative picture of colonies. Right: Quantification of colonies.

FIG. 7C. sGal-3 inhibits tumor growth. Six-week old female athymic nude mice (NCI) were injected subcutaneously with 5×10⁶ cells of clone #12 and divided into two groups (n=8). Similar results were obtained with clone #21. One group was left untreated while the second group was given 2 mg/ml Dox in drinking water containing 5% sucrose to induce expression of sGal-3 one week after tumor cell implantation until termination of the experiment. The length (a) and width (b) of the tumors were measured weekly and tumor volume was calculated in mm³=(a×b²)/2. No effect on tumor volume was observed in mice carrying LN229-L16 control tumors treated with dox over 12 weeks (data not shown).

FIG. 7D. Immunohistochemistry for Gal-3 expression in xenograft from mice in FIG. 7C. Representative tumor section showing that Gal-3 was expressed in tumors excised from mice treated with doxycycline.

FIG. 8: Model for p53 control switch over cell extrinsic Gal-3 apoptotic signalling. FIG. 8A. Cells with inactive p53 have low extracellular, and high cytoplasmic levels of Gal-3. The cytoplasmic Gal-3 is prevents apoptosis, possibly by blocking pro-apoptotic signalling at the mitochondrial membrane.

FIG. 8B. Activation of wt-p53 expression leads to transcriptional upregulation of TSAP6 expression and exosome formation. Cytoplasmic Gal-3 complexes with TSAP6 and is secreted in the tumor environment with a concomitant decrease in intracellular levels. The secreted Gal-3 binds to receptors on bystander cells leading to a caspase 3-dependent apoptotic response. Lightning bolt represents p53 activation stimulus.

FIG. 9. Representative 2-DE gels of extracellular proteins from glioma cells with inducible wt-p53 expression. FIG. 9A. Two p53-null clones (WT11 and 2024) with dox-inducible wt-p53 expression were used. Western blot shows wt-p53 induction, and downstream activation of the p21 cell cycle inhibitor, 48 hrs post induction in serum-free media.

FIG. 9B. Proteins found in the CM from uninduced (left) and wt-p53 induced (right) cells were analyzed by 2-DE analysis. Using IEF strips pH3-10 NL and 12.5% SDS-Page. Protein spots circled indicate proteins with enhanced secretion (right) or reduced (left) in response to wt-p53. Samples were run in triplicate and location of representative proteins is indicated. Black arrow shows p53-induced post-translational modification of Gal-1. White arrow shows the location of KIAA0828.

FIG. 9C. Enlargement showing acidic shift of Gal 1 in CM from wt-p53 expressing cells (arrows).

FIG. 10. Semi-quantitative analysis of differentially expressed proteins found by 2-DE and identified by MS/MS analysis. 3-D representation of differential expression for representative proteins found up-regulated (Gal-3, Gal-1, and β-2-microglobulin), down-regulated (SPARC, FGF-4, and TGF-beta), and unchanged (TSP-1 and pre-albumin) by 2-DE as analyzed by ImageMaster software.

FIG. 11. Comparative analysis of wt-p53 regulated extracellular proteins using both proteomic analyses. FIG. 11A. Ven Diagram showing the total number (111) of secreted proteins found by 2-DE (white), and cICAT (gray). FIG. 11B. Number of proteins found unchanged (white), up- (dark gray) or down-regulated (light gray) by wt-p53 expression using 2-DE and c-ICAT analysis alone and those common to. FIG. 11C. Distribution of identified secreted proteins by 2-DE (white) and c-ICAT (gray) analyses according to their general functional categories. Each protein is seen in a single category only even though some might play multiple functions.

FIG. 12. Verification of selected 2-DE and cICAT results. Western blot analysis on TCA-precipitated serum-free CM collected after 48 h from LNZ308-C16 (control for dox) and 2024 cells with tet-on wt-p53 expression. Differential expression of SPARC, FGF-4, TGF-beta, Gal-1, Gal-3 and β-2-M in response to wt-p53 expression was examined. TSP1 and pre-albumin were loading controls and remained unchanged.

### DETAILED DESCRIPTION OF THE INVENTION

The invention provides a method for preventing or treating cancer comprising administering a prevention or treatment effective amount of galectin, including but not limited to galectin-1 (Gal-1), galectin-3 (Gal-3), galectin-7 (PIG3), or other galectin or galectin-like molecules, to a patient in need thereof, thereby preventing or treating cancer via cancer cell apoptosis. The present invention also provides cell secretomes and/or secreted proteins whose extracellular levels are regulated by p53, i.e., p53-mediated secretomes. As used herein, the term “secretome” refers to proteins released through classical as well as non-classical secretion pathways (Volmer et al. 2005). In addition, the term “secretome” also includes intracellular proteins and protein fragments that might be released in exosomes as a result of wt-p53 expression. As used herein, the invention contemplates the use of the secretome component proteins, and homologs, analogs and orthologs thereof. As used herein, the term “downstream effector” refers to a protein which responds to p53 signaling to effect a change in the secretome, such as but not limited to TSAP6.

In preferred embodiments, the present invention provides that p53 can exert cell-extrinsic control over tumor growth through the modulation of extracellular levels of secretomes, preferably Gal-1 or Gal-3, more preferably, Gal-3. In particular, the present invention provides that p53 expressing cells can induce cell death in bystander cells through p53 controlled extracellular levels of Gal-3, and the use of soluble Gal-3 for cancer treatment. The present invention further provides that Gal-3 extracellular levels can be facilitated by p53 transcriptional activation of TSAP6, a key mediator of the non-traditional secretory pathway. The biological importance of p53 controlled Gal-3 secretion is demonstrated herein by showing that Gal-3 inhibits anchorage-independent tumor cell growth in vitro and strongly reduces tumor formation in vivo. Therefore, the invention provides that secretome components, such as Gal-3 itself, or as the result of the modulation of its expression by p53 or a p53 downstream effector, such as TSAP6 can be used for cancer treatment or other diseases mediated by extracellular protein signaling.

In a preferred embodiment, the present invention provides enhanced extracellular levels of the pro-apoptotic factor Gal-3 for therapeutic uses. The present invention further provides that the mechanism mediating p53 control over Gal-3 extracellular levels involves transcriptional regulation of TSAP6, a transmembrane protein that plays a key role in protein secretion by exosomes, and activation of p53 in cells induce paracrine killing effects in bystander cells. The regulation by p53 of the anti-tumor activity of extracellular Gal-3 represents a new paracrine pro-apoptotic and tumor suppressive function of p53 and provides a novel molecular mechanism for p53-mediated bystander effects in anti-cancer treatments. This bystander effect represents a novel tumor suppressive mechanism for p53 and is important for p53 gene therapy and chemo- and radiotherapies.

The present invention further provides methods of determining the diagnosis or prognosis of an individual by detecting extracellular levels of Gal-3, or other p53 mediated secretome components expressed and/or secreted, in the individual as compared to levels of expression and secretion in not mal individual. The present invention provides a way to adjust the therapy regimen based on measurements in the patient of secreted Gal3 and p53 and TSAP6 levels.

In certain preferred embodiments, the present invention provides a new indirect target of the p53 tumor suppressor that can influence tumor growth. For example, the present invention provides enhanced extracellular levels of the pro-apoptotic factor Gal-3, and further provides that the mechanism mediating p53 control over extracellular levels of Gal-3 involves transcriptional regulation of TSAP6. The present invention provides that Gal-3 co-localizes with and interacts with TSAP6. Up-regulation of TSAP6 expression in cells leads to Gal-3 relocation to the cell membrane and secretion through exosomes. Extracellular levels of Gal-3 can induce autocrine and paracrine cell death and reduction of tumor growth. The regulation by p53 of the anti-tumor activity of extracellular Gal-3 represents a new paracrine pro-apoptotic and tumor suppressive function of p53 and provides a novel molecular mechanism for p53-mediated bystander effects in anti-cancer treatments.

The present invention provides p53's control over tumor growth through the extracellular levels of pro-apoptotic Gal-3 provides a critical link between ectocytosis and the tumor suppressive role of p53. The present invention thus provides an important aspect for the design and understanding of chemo- and radiotherapy regimens for cancer as well as for p53 gene therapy. Adjusting the therapy dose and schedule according to patient Gal3 expression and secretion levels and p53 and TSAP6 expression are implied.

The present invention further provides that the soluble form of Gal-3 had a cytotoxic effect in vitro on a variety of cancer cell lines from different organs, due to the induction of caspase-mediated apoptosis, even in those lines resistant to other pro-apoptotic signals or carrying genetic defects in apoptotic pathways (Hao et al. 2001; Song et al. 2003). These findings may in part explain previous observations on the induction of apoptosis by TSAP6 (Zhang et al. 2001; Passer et al. 2003; Porkka et al. 2003). Wt-p53 control over a secreted pro-apoptotic factor suggested that paracrine-mediated cell killing may occur. The present invention demonstrated this cell killing in cell mixing experiments where activation of p53 in UV, gamma irradiation or chemotherapy treated cells induces the killing of adjacent p53 null cells. This paracrine killing mechanism may contribute to the “bystander effects” observed with chemotherapy and in p53 gene therapy clinical trials (Little et al. 2002; Fang and Roth 2003) and suggest that restoration of p53 function in part of the tumor may have therapeutic effects beyond the transduced cells. The present invention further provides that sGal-3 was a strong inhibitor of tumor growth in nude mice. Therefore, the present invention provides Gal-3 to be used for cancer therapy, as cancer cells are more sensitive to its apoptotic effects than normal cells.

The present invention provides a means to produce efficiently high amounts of sGal3 in 293 human cells. Sgal3 is produced from an expression vector that contains an engineered Gal3 gene that incorporates a classical secretion signal. The cells are transfected with this construct and high levels are collected in the cell supernatant without intracellular accumulation (FIG. 6A).

The present invention and those derived from the literature documenting an anti-apoptotic role of Gal-3 at the mitochondrial membrane can be incorporated in the following non-limiting working model (FIG. 8). TSAP6 recruits the cytoplasmic fraction of Gal-3 to the cell membrane where it is packaged in exosomes for ectocytosis. This results in depletion of the cytoplasmic pool of Gal-3 and therefore, indirectly inhibits the anti-apoptotic function of Gal-3 located in the cytoplasmic space. By modulating TSAP6, p53 would act as a molecular switch for intracellular versus extracellular Gal-3 localization, effecting the transition from an anti- to a pro-apoptotic function.

While the mechanism just described may contribute per se to the pro-apoptotic effects induced by augmented extracellular levels of Gal-3, it does not account for the induction of apoptosis by exposure to exogenous Gal-3. The latter observation indicates that extracellular Gal-3 binds to a ligand on the cell surface, which leads directly to the induction of the observed apoptotic response.

The present invention also provides methods of identifying and screening p53 regulated cell secretomes, and methods for the modulation of the composition of secreted extracellular proteins, or the secretome. The present invention provides that p53 expression, or other means of controlled administration of p53, can be used to effect the relative composition of the secretome. The present invention also provides that other downstream effectors of p53, such as TSAP6, may be used as the modulator of secreted extracellular protein. The present invention is useful for modulating the secretome of a cell, or in a patient generally, to prevent or treat diseases mediated by intercellular communication, such as tumorigenesis, angiogenesis, apoptosis, and immune-related disorders. Therefore, in certain embodiments, the present invention provides compositions and methods useful for modulating the secretome of a cell, comprising modulating a secretome component, such as Gal-3, by direct administration of the p53 mediated secretome component, or by regulating p53 expression, or expression of a downstream effector of p53, in the cell.

In preferred embodiments, the secretome modulation enhances stability and/or secretion pathways of the protein. In certain preferred embodiments, the modulation can be post-translational, including modifications affecting glycosylation, phosphorylation, or hydroxylation, which can affect the half-life, trafficking or function of the protein. In certain preferred embodiments, the modulation can related to improving secretion, such as by increasing protein secretion through non-classical pathways, such as exocytosis, ectocytosis and transporter-mediated protein secretion. In certain preferred embodiments, the modulation can affect the regulation of tumor invasion and metastasis via induction of structural and pro-adhesion molecules used in cell-cell and cell-matrix interaction, and effect anti-migratory factors.

Therefore, in certain embodiments, the present invention provides a useful tool for screening for other effectors of the secretome which inhibit or enhance the novel regulatory systems disclosed herein. Furthermore, in certain embodiments, the present invention provides and evaluates values of cell secretomes and/or proteins identified by the present invention as diagnostic and prognostic indicators and for disease treatment and treatment follow-up. Moreover, in certain embodiments, the present invention provides development and identification of biomarkers in tumor biology, and use in the clinic, as well as for therapeutic targets for cancer therapies.

Therefore, the present invention provides methods for modulating an extracellular secretome comprising controlling expression of p53, or a downstream effector of p53, in a cell or in a patient in need thereof, thereby modulating the extracellular secretion of said secretome.

Furthermore, the present invention provides methods for identifying a biomarker or a tumor target comprising determining secretion levels of a secreted protein in the presence and absence of wt-p53 expression, comparing the secretion levels of said secreted protein in normal cells and cancer cells, and identifying said secreted proteins having extracellular secretion levels regulated by wt-p53 in cancer cells, thereby identifying the biomarker or the tumor target.

The invention also provides methods for determining a diagnosis or a prognosis of a disease state comprising determining in a patient extracellular secretion of a protein regulated by p53 expression associated with a disease, thereby determining the diagnosis or the prognosis of the disease state. The invention provides methods for preventing or treating a disease comprising administering a treatment effective amount of p53, or a downstream effector of p53, to a patient in need thereof to regulate extracellular protein secretion associated with the disease, thereby preventing or treating the disease. Target patient populations could be the tumor-prone patients carrying germline p53 mutations (Li-Fraumeni syndrome) or patients carrying a tumor that has lost p53 function due to somatic genetic alterations.

The invention further provides compositions comprising such identified biomarkers and tumor targets and compositions for achieving the above methods of modulation, diagnosis, prognosis, prevention and treatment of disease states. In certain embodiments, the disease is cancer-related, angiogenesis-related, or immunology-related.

The inventions provided herein can be practiced using conventional molecular biology and protein isolation and purification methods known in the art, such as described in Sambrook, et al., Molecular Cloning: A Laboratory Manual. latest ed., Cold Spring Harbor Laboratory, Cold Spring Harbor Laboratory Press, Cold Spring Harbor, N.Y., 1989 (current edition). In addition, the compositions and methods for modulation of the secretome, diagnosis, prognosis, prevention and treatment of disease can be practiced using the level of ordinary skill in the art of medicine depending upon such variable factors as the age, sex, predisposition and condition of the patient and state of the disease.

As stated above, the present invention contemplates the use of the secretome component proteins, which are intended to include functional equivalents such as homologs, analogs and orthologs thereof. In preferred embodiments, the present invention contemplates secretome component proteins, including galectin family, preferable Gal-3, their homologs, analogs and orthologs. As used herein, the term “analogs” refers to two amino acids that have the same or similar function, but that have evolved separately in unrelated organisms. As used herein, the term “analog” further refers to a structural derivative of a parent compound that often differs from it by a single element. As used herein, the term “analog” also refers to any peptide modifications known to the art, including but are not limited to changing the side chain of one or more amino acids or replacing one or more amino acid with any non-amino acids.

As used herein, “homologs” are defined herein as two nucleic acids or peptides that have similar, or substantially identical, nucleic acids or amino acid sequences, respectively. The term “homolog” further encompasses nucleic acid molecules that differ from one of the nucleotide sequences due to degeneracy of the genetic code and thus encodes the same amino acid sequences. In one of the preferred embodiments, homologs include allelic variants, orthologs, paralogs, agonists, and antagonists of Gal-3.

As used herein, the term “orthologs” refers to two nucleic acids from different species, but that have evolved from a common ancestral gene by speciation. Normally, orthologs encode peptides having the same or similar functions. In particular, orthologs of the invention will generally exhibit at least 80-85%, more preferably 85-90% or 90-95%, and most preferably 95%, 96%, 97%, 98%, or even 99% identity, or 100% sequence identity, with all or part of the amino acid sequence of Gal-3 or analogs thereof and will exhibit a function similar to these peptides. As also used herein, the term “paralogs” refers to two nucleic acids that are related by duplication within a genome. Paralogs usually have different functions, but these functions may be related (Tatusov et al., 1997, Science 278(5338):631-637).

The percent sequence identity between the two sequences is a function of the number of identical positions shared by the sequences (i.e., percent sequence identity=numbers of identical positions/total numbers of positions×100). Preferably, the isolated amino acid homologs included in the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence of Gal-3. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence encoding amino acid sequences of Gal-3. It is further preferred that the isolated nucleic acid homologs of the present invention encode amino acid sequences of Gal-3, respectively, or portion thereof, that is at least 90%, more preferably at least 95% identical to an amino acid sequence of Gal-3.

The determination of the percent sequence identity between two nucleic acid or peptide sequences is well known in the art. For instance, the Vector NTI 6.0 (PC) software package (InforMax, 7600 Wisconsin Ave., Bethesda, Md. 20814) to determine the percent sequence identity between two nucleic acid or peptide sequences can be used. In this method, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Using the above-described methods, and others known to those of skill in the art, one of ordinary skill in the art can isolate homologs of Gal-3. One subset of these homologs are allelic variants. As used herein, the term “allelic variant” refers to a nucleotide sequence containing polymorphisms that lead to changes in the amino acid sequences of Gal-3 without altering the functional activities. Such allelic variations can typically result in 1-5% variance in nucleic acids encoding Gal-3.

In addition, the skilled artisan will further appreciate that changes can be introduced by mutation into a nucleotide sequence that encodes the amino acid sequence of Gal-3, or analogs thereof. For example, nucleotide substitutions leading to amino acid substitutions at “non-essential” amino acid residues can be made in a sequence encoding the amino acid sequence of Gal-3 or analogs thereof. A “non-essential” amino acid residue is a residue that can be altered without altering the activity of said peptide, whereas an “essential” amino acid residue is required for desired activity of such peptide.

Accordingly, another aspect of the invention pertains to nucleic acid molecules encoding Gal-3 that contain changes in amino acid residues that are not essential for its activity. In one embodiment, the isolated nucleic acid molecule comprises a nucleotide sequence encoding peptide, wherein the peptide comprises an amino acid sequence at least about 50% identical to an amino acid sequence of Gal-3. Preferably, the peptide encoded by the nucleic acid molecule is at least about 50-60% identical to an amino acid sequence of Gal-3, more preferably at least about 60-70% identical, even more preferably at least about 70-75%, 75-80%, 80-85%, 85-90%, or 90-95% identical, and most preferably at least about 96%, 97%, 98%, or 99% identical to an amino acid sequence of Gal-3.

An isolated nucleic acid molecule encoding a polypeptide having amino acid sequence identity to Gal-3 can be created by introducing one or more nucleotide substitutions, additions, or deletions into a nucleotide encoding peptide sequence of Gal-3, respectively, such that one or more amino acid substitutions, additions, or deletions are introduced into the encoded peptide and/or the side chain of the amino acids constituting the encoded peptides. Mutations can be introduced into the nucleic acid sequence encoding the peptide sequence of Gal-3 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Thus, a predicted nonessential amino acid residue in Gal-3 is preferably replaced with another amino acid residue from the same side chain family. Alternatively, in another embodiment, mutations can be introduced randomly along all or part of a peptide sequence for Gal-3, such as by saturation mutagenesis, and the resultant mutants can be screened for Gal-3 activity described herein. Following mutagenesis of the nucleic acid sequence encoding the peptide sequence of Gal-3, the encoded peptide can be expressed recombinantly and the activity of the peptide can be determined. In this way variants of Gal3 with enhanced activity may also be derived.

The nucleotides of the present invention may be produced by any means, including genomic preparations, cDNA preparations, in vitro synthesis, RT-PCR, and in vitro or in vivo transcription. The design and production of nucleic acids encoding a desired amino acid sequence is well known to those of skill in the art based on standardized codons. In preferred embodiments, the codons selected for encoding each amino acid may be modified to optimize expression of the nucleic acid in the host cell of interest. Codon preferences for various species of host cell are well known in the art.

The present invention further provides a composition comprising at least a secretome whose extracellular secretion is regulated by p53, and at least one pharmaceutically active agent or drug in an effective amount to provide desired pharmaceutical effects, either locally or systematically. As stated above, the secretome provided in the composition comprise Gal-3 and/or other secretome components in response to p53 expression. In one of the preferred embodiments, the present invention provides a therapeutic composition comprising Gal-3 or analogs thereof.

The present invention further provides a composition comprising Gal3 with different post-translational modifications. These may be generated by production of Gal3 through physiological induction of secretion in different cell types, through expression of a genetically engineered gene with an altered secretory signal or produced in other organisms (bacteria, insect cells, etc.).

As used herein, the term “pharmacologically active agent,” “therapeutic agent,” “active agent,” or “drug” is used interchangeably to refer to a chemical material or compound that induces a desired pharmacological, physiological effect, and include agents that are therapeutically effective, prophylactically effective, or cosmeceutically effective. The terms also encompass pharmaceutically acceptable, pharmacologically active derivatives and analogs of those active agents specifically mentioned herein, including but are not limited to, salts, esters, amides, prodrugs, active metabolites, inclusion complexes, analogs, and the like. When the terms “pharmacologically active agent,” “active agent,” and “drug” are used, it is to be understood that applicants intend to include the active agent per se as well as pharmaceutically acceptable, pharmacologically active salts, esters, amides, prodrugs, active metabolites, inclusion complexes, analogs, etc., which are collectively referred to herein as “pharmaceutically acceptable derivatives”.

As used herein, the pharmaceutically active agents also include any vectors/virus used for gene therapy. The term “gene therapy” refers to a technique for correcting defective genes responsible for disease development and the use of any gene transfer for therapeutic purposes (without necessarily the purpose of replacing an underlying defective one). Such techniques may include inserting a normal gene into a nonspecific location within the genome to replace a nonfunctional gene; swapping an abnormal gene for a normal gene through homologous recombinations, repairing an abnormal gene to resume its normal function through selective reverse mutation; and altering or regulating gene expression and/or functions of a particular gene. In most gene therapy, a normal gene is inserted into the genome to replace an abnormal or disease-causing gene or to increase expression of a particular protein in a tissue that naturally does not express it or expresses it at low levels. As used herein, a term “vector/virus” refers to a carrier molecule that carries and delivers the “normal” therapeutic gene to the patient's target cells. Because viruses have evolved a way of encapsulating and delivering their genes to human cells in a pathogenic manner, most common vectors for gene therapy are viruses that have been genetically altered to carry the normal human DNA. As used herein, the viruses/vectors for gene therapy include retroviruses, adenoviruses, adeno-associated viruses, and herpes simplex viruses. The term “retrovirus” refers to a class of viruses that can create double-stranded DNA copies of their RNA genomes, which can be further integrated into the chromosomes of host cells, for example, Human immunodeficiency virus (HIV) is a retrovirus. The term “adenovirus” refers to a class of viruses with double-stranded DNA genomes that cause respiratory, intestinal, and eye infections in human, for instance, the virus that cause the common cold is an adenovirus. The term “adeno-associated virus” refers to a class of small, single-stranded DNA viruses that can insert their genetic material at a specific site on chromosome 19. The term “herpes simplex viruses” refers to a class of double-stranded DNA viruses that infect a particular cell type, neurons. Herpes simplex virus type 1 is a common human pathogen that causes cold sores.

The pharmaceutically active agents as used herein also refer to vaccines that comprise a suspension of attenuated or killed microorganism (e.g. bacterial, viruses, or ricjettsiae) that are administered for the prevention, amelioration or treatment of infectious diseases. As used herein, the term “vaccine” refers to a product that produces immunity therefore protecting the body from the disease. Currently, vaccines are administered through needle injections, by mouth and by aerosol. As used herein, any vaccines currently available in the art and any vaccines in the development stage are within the scope of the present invention.

As used herein, the term “effective amount” or “therapeutically effective amount” of a pharmaceutically active agent is intended to mean a nontoxic but sufficient amount of a pharmaceutically active agent to provide the desired therapeutic effect. The amount that is effective will vary from subject to subject, depending on the age and general condition of the individual, the particular active agent or agents, and the like. Thus, it is not always possible to specify an exact effective amount. However, an appropriate effective amount in any individual case may be determined by one of ordinary skill in the art using routine experimentation. Furthermore, the exact effective amount of an active agent incorporated into a composition or dosage form of the present invention is not critical, so long as the concentration is within a range sufficient to permit ready application of the solution or formulation so as to deliver an amount of the active agent that is within a therapeutically effective range. As used herein, the composition can be made in any of formulations suitable for administration, and may be administered in any desired route of administration.

In preferred embodiments, the wt-p53 regulated secreted proteins identified herewith are not transcriptional targets and the invention provides that p53 has an indirect role in their stability or secretion. The present invention also provides that p53 loss in tumors acts as an originator of changes in tumor-stroma interactions.

In certain embodiments, the present invention provides regulation of p53 on the cell's secretome by identifying secreted proteins using p53-null tumor cells in the presence or absence of reconstituted wt-p53 expression. The present invention provides a comprehensive analysis of how p53 plays a role in the process of transformation through its manipulation of the tumor microenvironment. Many of the p53 regulated secreted proteins identified by the present invention have known roles in cancer-related processes that are dependent on heterotypic cell-cell communication such as immune response, angiogenesis, extracellular matrix interaction and cell survival, and are secreted through receptor mediated classical and/or non-classical secretory pathways. The present invention provides an important and advanced understanding on how tumor-stroma interactions contribute to cancer progression.

The present invention further provides a comprehensive analysis of the tumor cell secretome to identify secreted targets of wt-p53. The mechanism through which p53 might regulate the secretion of proteins is heretofore unknown. A number of secreted proteins regulated at the trancriptional level have been reported. However, the secreted proteins provided by the present invention are not significantly regulated by p53 at the transcriptional level (Harada et al. 2003, Khwaja et al, 2006).

The present invention further provides that p53 enhances stability of these secreted proteins, such as through down-regulation of proteases like MMPs. Alternatively, the present invention provides that p53 alters the secretion rate of intracellular proteins through either augmented release of specific proteins or through upregulation of a particular secretory pathway, thus leading to enhanced levels of all proteins secreted through that pathway. It has been reported that the p53-regulated protein, TSAP6, can facilitate the secretion of another protein (TCTP) through ectocytosis (Amzallag et al. 2004).

The present invention further provides functional implications for tumorigenesis. Wt-p53 has been shown to inhibit many processes required for tumor growth including migration, angiogenesis, survival and cell proliferation (Fulci and Van Meir 1999). It has also been implicated in eliciting an immune response against neoplastic cells (Hoglund 2006). The present invention provides that p53 regulates the secretion of many proteins that are candidate mediators for the above biological effects.

p53 and metastasis/invasion. The present invention provides that several extracellular matrix (ECM) components, including but not limited to growth arrest-specific 6, collagen type XI alpha-1, proteoglycan PG-M, or proteins involved in adhesion and cell-matrix interactions, including but not limited to galectin-3, lysyl oxidase-like protein 2, osteopontin, alpha-catenin and beta-5 tubulin, as well as protease inhibitors, including but not limited to TIMP-3 and glioma pathogenesis-related protein, are up-regulated in the CM from the glioma cells after p53 induction. The induction of these structural and pro-adhesion proteins would be expected to improve cell-cell and cell-matrix interactions, thus resulting in reduced migratory potential of tumor cells. In addition to upregulation of anti-migratory factors, the present invention also provides that other proteins directly involved in induction of migration and invasion in multiple tumor types are down-regulated by p53. These include but are not limited to SPARC, MMP-2, TGF-beta, ADAM-10 and Tau (Framson and Sage 2004; Mazzocca et al. 2005; Stuelten et al. 2005). The present invention provides a potential new facet of p53's multimodal function as a tumor suppressor gene, through down-regulation of tumor invasion and metastasis.

p53 and the immune response. In recent years various studies have suggested that wt-p53 could stimulate immune responses against tumor cells. For example, secreted osteopontin (Opn) found upregulated in the present invention is one of the key cytokines for type 1 immune responses mediated by macrophages. It has already been reported as a direct target of wt-p53 and has been implicated in suppressing tumor growth in vivo (Morimoto et al. 2002). The present invention also provides increased secretion of immune response related proteins beta-2-microglobulin (β-2M) and macrophage migration and myeloid leukemia inhibitory factors in response to p53. β-2M is a MHC class I molecule and several studies have shown that tumor development might be inhibited by immune responses stimulated by this class of proteins (Bueter et al. 2006). Other immune related proteins like interleukin-8 (IL-8), attractin and ANP32A are also down-regulated by p53. Interleukin-8 is known to be upregulated in glioma, possibly in response to immune cell infiltration (Desbaillets et al. 1997). Attractin is upregulated in glioma patient CSF and can modulate T cell motility (Khwaja et al. 2006 Clin Cancer Res 12, 6631).

p53 and angiogenesis. The present invention further provides repression of certain pro-angiogenic proteins, including but not limited to VEGF, IL-8, transforming growth factor beta, PEDF and CYR61 by p53. VEGF has been shown to be down-regulated by wt-p53 in many systems (Miyagami and Katayama 2005) while CYR61 has not been reported as a p53 target before. CYR61 is a secreted extracellular matrix-associated signaling molecule that has been shown to promote the adhesion and proliferation of endothelial cells (Babic et al. 1998). CYR61 has been shown to be overexpressed in several cancers including breast and brain tumors, where it promotes angiogenesis and increased tumor growth (Tsai et al. 2000; Xie et al. 2004). Similarly, IL-8 is expressed and secreted at high levels in human gliomas and is critical to glial tumor neovascularity and progression (Brat et al. 2005). The present invention provides that p53 loss in tumors activates angiogenesis by an increase in secretion of pro-angiogenic factors and decrease of inhibitors.

p53 and tumor proliferation and survival. The present invention further provides that several proteins regulating tumor proliferation and survival are regulated by wt-p53. For instance, brain derived neurotrophic factor (BDNF) exhibited enhanced secretion in response to p53. The secreted form of BDNF mediates apoptosis of cells containing neurotrophin receptors (Lee et al. 2001). Other proteins, including FGF-4, RTVP1, TPM-ALK fusion oncoprotein fragment, TGF-β, PEDF, IGFBPs and granulin were all found to have inhibited secretion to varying degrees in response to wt-p53. RTVP1, TGF-β (Tsuzuki et al. 1998) and PEDF (Pietras et al. 2002) are previously known targets of wt-p53.

Accordingly, the present invention identifies a cell-extrinsic mechanism by which p53-expressing cells control the growth of adjacent cells. The present invention further provides that p53 controls the secretion of pro-apoptotic factors that exert autocrine and paracrine growth suppressive effects on tumors by inducing tumoral cell and stromal cell death. Furthermore, the present invention provides mediators of the p53-induced bystander effect by screening for p53-regulated proteins in the secretome of human tumor cells for pro-apoptotic factors (Khwaja et al. 2006).

These and many other variations and embodiments of the invention will be apparent to one of skill in the art upon a review of the appended description and examples.

### EXAMPLES

### Example 1

### Paracrine Induction of Apoptosis Through Galectin-3 Secretion

### Experimental Procedures:

### Cell Lines:

Human glioblastoma cell lines LN-Z308 (p53 null) parental (Albertoni et al. 1998), and derived clones LN-Z308-C16 (rtTA expressing), 2024 (tet-on inducible wt-p53) (Albertoni et al. 2002), and WT11 (tet-off inducible wt-p53) (Van Meir et al. 1994) have been previously described. Glioma cell lines LN229, clone LN229-L16 (rtTA expressing) (Kaur et al. 2005), U87MGD and SF767 (Ishii et al. 1999), 293CLH (embryonic kidney cells), breast cancer cell lines (MCF7 and MD468), lung cancer cell lines (A549 and H1289), and prostate cancer cell lines (PC3 and LnCaP), human foreskin fibroblasts (HFF) and human dermal microvascular endothelial cells (HDMEC) were grown in DMEM supplemented with 5% tet-free FCS (Gibco; NY USA). HCT116 human colon carcinoma cell lines were grown in McCoy media supplemented with 5% FCS. Cells were plated in 150 cm² plates at 60% confluency and allowed to grow overnight.

The following day cells were switched to serum-free media after three washes with DPBS without calcium and magnesium. Wt-p53 or sGal-3 expression was induced by modulation with 2 μg/ml of doxycycline (dox) for 48 h and Conditioned media (CM) was collected from the cells. Floating cells were removed from the CM by centrifugation at 1,000 g and it was either frozen at −20° C. or used undiluted on target cells in cell viability assays. For cell mixing experiments, p53 null and wt-p53 expressing cells were labeled with red and green fluorescent days respectively (Cell Tracker dyes; Invitrogen, USA). Neutralization of Gal-3 in CM was achieved by addition of 100 μg of anti-Gal-3 antibody per 1 ml of media. Wt-p53 activation was achieved by treating the cells with 25 μM of etoposide (VP16),

### Two-Dimensional Gel Electrophoresis (2-DE):

Extracellular proteins present in CM were TCA precipitated and resuspended in 8 M urea, 4% Chaps, 100 mM Protease inhibitor cocktail (Roche, Germany). 2-DE was performed as described before and galectin levels were estimated using image-master software on silver stained gels as well as 2-D western analyses (Khwaja et al. 2006).

### Protein Extraction and Western Blot Analysis:

Immunoblots were performed on cell lysates (lysed in 8 M urea, 4% SDS, in 10 mM Tris (pH 7.4)), from indicated cells. The conditioned media was precipitated by 15% TCA precipitation for 2 h at 4° C., washed twice with ice-cold acetone, and then resuspended in lysis buffer (8M urea, 4% SDS, 100 mM protease inhibitor cocktail (Roche, Germany)). Electrophoresis and blotting was performed using the Criterion system (BioRad; CA, USA) on. TCA precipitated proteins. Western blots were probed with antibodies for p53 (M7001-clone DO-7; DAKO; 1:500), p21 (MS-891-P1; Neomarkers, CA, USA; 1:250), Gal-3 (ab14364; Santa Cruz, Calif., USA; 1:500), HA (Roche; Germany, 1:500), caspase-3 (1:5000), α-TSP1 (MS-421-P1-Ab-4; NeoMarkers; CA, USA; 1:1000), and PARP (556494; BD Biosciences; CA, USA, 1:1000). Actin was used as a loading control (sc-1615, Santa Cruz Biotechnology; CA; 1:500). Blots were visualized by enhanced chemiluminescence (Pierce, IL, USA). For apoptosis analysis, cells were treated with either media containing secreted Gal-3 or with both secreted Gal-3 and 50 μm Z-VAD-FMK general caspase inhibitor (BD Biosciences; CA, USA).

### Northern Blot Analysis:

Total RNA was isolated using the Trizol method (Invitrogen; CA, USA). Pairs of primers were used for each gene of interest to generate probes for Northern blot analysis by reverse transcription-PCR amplification. The sense and the antisense primers were 5′-AGAGGTTCAAGCGATTCTCCTGCT-3′ (SEQ ID NO:1) and 5′-TGCTGAAGGTGCTCTTGCTCTGTA-3′ (SEQ ID NO:2) (TSAP6); 5′-AGATTATATCATGGTATATGAAAG-3′ (SEQ ID NO:3) and 5′-AGATTATATCATGGTATATGAAAG-3′ (SEQ ID NO:4) (Galectin-3); 5′-CCTGCCCTCAACAAGATGTT-3′ (SEQ ID NO:5) and 5′-GGTGAGGCTCCCCTTTCTTG-3′ (SEQ ID NO:6) (p53); 5′-GGTACAAGACAGTGACAGGTC-3′ (SEQ ID NO:7) and 5′-GTTCCTTGTGGAGCCGGAGC-3′ (SEQ ID NO:8) (p21); 5′-TGAAGGTCGGAGTCAACGGATTTGGT-3′ (SEQ ID NO:9) and 5′-CATGTGGGCCATGAGGTCCACCAC-3′ (SEQ ID NO:10) (GAPDH). RNA was resolved on 1% agarose-formaldehyde gels and transferred to nylon membranes as described (Tan et al. 2005). The hybridization was done with the reverse transcription-PCR-generated cDNA probes specific for human Galectin-3, TSAP6, p53, p21 and glyceraldehyde-3-phosphate dehydrogenase (GAPDH). Probes were labeled with [α-³²P] dCTP (Amersham Biosciences; NJ, USA) using Prime-It II Random Primer Labeling Kit (Stratagene; TX, USA). Hybridization was carried out overnight as described using the ULTRAhyb buffer (Ambion, TX, USA) at 42° C. (Tan et al 2005).

### Expression Vectors and Transfections:

The 753 by Gal-3 cDNA was amplified by RT-PCR from SF767 glioma cells, sequence verified and cloned into the XbaI cloning site of the pcDNA3.1 myc His expression vector (Invitrogen; CA, USA). Similarly, Gal-3 cDNA without the start codon was amplified and cloned into the EcoR1 site of pUMVC7 plasmid (Aldevron; ND, USA) containing a classical secretion signal peptide from the tissue plasminogen activator, to generate plasmid pUMVC7-sGal3 that constitutively secretes Gal-3 (sGal-3) from a CMV promoter. To construct tetracycline-inducible secreted Gal-3, the fragment containing sGal-3 (secretion signal plus Gal-3 transcript) was excised from this plasmid with BssHII and Not 1, and cloned into the pTRE2 expression vector (Clontech; CA, USA). The HA-tagged TSAP6 containing pcDNA3.1 plasmid has been described previously (Amzallag et al. 2004). All transfections were carried out by plating 2×10⁶ cells/10 cm plates 24 h prior to transfection.

Five μg of the plasmid DNA and 5 μl of GenePORTER reagent (Gene Therapy Systems; San Diego, Calif.) were used for each transfection. One μg of pcDNA3.1 containing the geneticin (G418)-resistance cassette was co-transfected at a 1:25 ratio, when generating stable clones. Tetracycline-inducible sGal-3 transfected clones were generated in L16 glioma cells (Tet-on clone derived from LN229) by selecting for G418 resistance (1,200 μg/ml). The individual clones were assessed for expression of sGal-3 after 48 h dox induction by western blot analysis. For TSAP6 siRNA, HEK293 cells were transfected with either pcDNA3.1 vectors containing TSAP6 and Galectin-3 alone or in combination with 50 control or TSAP6 (pHYDE) siRNA (Santa Cruz, Calif., USA) using GenePORTER reagent (Gene Therapy Systems; CA, USA). Cells were allowed to recover overnight in 10% FBS containing DMEM before switching them to serum free media. Cells and CM were collected after 24 hours in serum free media.

### Co-Immunoprecipitation of TSAP6 and Gal-3:

Human 293CLH cells or HeLa cervical carcinoma cells were transfected with expression vectors for Gal-3, TSAP6-HA or pCMV-lacZ plasmid as a control. The transfected cells were switched to serum free media and the cells and CM were collected 48 h later. The cell extracts were prepared in RIPA lysis buffer and the protein concentration was calculated using RCDC protein concentration assay (BioRad; CA, USA). One milligram of protein extracts from each condition were immunoprecipitated overnight at 4° C. using 20 μl protein A/G agarose beads (se-2003, Santa Cruz, Calif., USA) and 2 mg of either α-HA or α-Gal-3 antibody. After overnight incubation, the beads were collected by centrifugation at 5,000 g and washed twice with RIPA buffer. The immunoprecipitated proteins were then solubilized in SDS loading buffer with 0.5% beta-mercaptoethanol. The samples were heated for 5 min at 100° C. before western analysis. Each set of immunoprecipitated sample was loaded in duplicate and the western was probed with both α-Gal-3 and α-HA antibodies.

### Co-Immunofluorescence Staining:

HeLa or HEK293-CLH cells were cotransfected with plasmids containing Gal-3 and HA-tagged TSAP6 cDNAs using GenePORTER reagent (Gene Therapy Systems; CA, USA). The cells were allowed to recover overnight, then trypsinized and plated onto cell culture treated slides (138121; Nunc, IL, USA). After overnight growth, the cells were washed twice with PBS, fixed with 4% formaldehyde and permeabilized with 0.2% Triton-X 100. Following permeabilization, cells were incubated sequentially for αGal-3 (goat anti-Gal-3 (1:500; Santa Cruz, Calif., USA) primary antibody followed by donkey anti-goat Alexa Fluor 594 (1:2000; A11008; Molecular probes, USA) and αTSAP6 (rabbit anti-HA (1:500; Santa Cruz, Calif., USA) followed by goat anti-rabbit Alexa Fluor 488 (1:2000; A11009; Molecular probes, USA) respectively. Following antibody staining, the nuclei were stained with Hoescht staining (1:10,000) and the slides were covered with anti-fade prolong Gold mounting medium and a cover slip.

### MTT Proliferation and Crystal Violet Cytotoxicity Assays:

293 cells were transfected using GenePORTER reagent (Gene Therapy Systems; CA, USA) with the pUMVC7-sGal-3 plasmid and switched over to serum free media 16 h later. The CM was collected after 48 h, floating cells removed through centrifugation at 1000 g, and filtrated through a 0.8 μm filter (Corning; NY, USA). Cells were plated at 5,000 cells per well in 24-well plates and treated with serial dilutions of CM from either pCMV-LacZ or pUMVC7-sGal-3 transfected 293 cells. Relative cell number compared to serum free media control was quantified in triplicate 24 h and 48 h post treatment using either an MTT kit (Roche; IN, USA) or by crystal violet assay by acquiring absorbance at 575 nm using a spectrophotometer.

### Soft-Agar Colony Formation Assays:

The LN229-L16 parental cells along with a low, medium and a high expressing tet-on sGal-3 clone were used to examine anchorage independent cell growth using a soft-agar colony formation assay. 6-well plates were layered with 2 ml of 1% agar in DMEM medium supplemented with 10% tet-free serum. This bottom layer was overlaid with 5,000 cells mixed in with 0.33% agar with DMEM and 10% tet-free serum. One ml of 10% tet-tested serum containing media with or without 5 μg/ml of dox to induce expression of secreted galectin-3 was added to the top every 72 h. The cells were allowed to grow and form colonies for 21 days. The colonies were then fixed using 100% methanol and visualized using Giemsa stain according to the manufacturer's protocol (Sigma). The plates were air-dried to flatten the agar discs, the colonies counted and photographed at 20×. The entire experiment was repeated three times in triplicate with reproducible results.

### In Vivo Mouse Tumorigenicity Assays:

Six-week old female athymic nude mice (NCI) (n=8/group) were injected subcutaneously with 5×10⁶ cells of the indicated cell lines. 2 mg/ml Dox was delivered orally in drinking water containing 5% sucrose to induce expression and secretion of Gal-3 one week post injection of tumor cells until termination of the experiment. The length (a) and width (b) of the tumors were measured weekly and tumor volume was calculated in mm³=(a×b²)/2.

### Results:

### Wt-p53 Activated Cells can Kill p53 Null Bystander Cells:

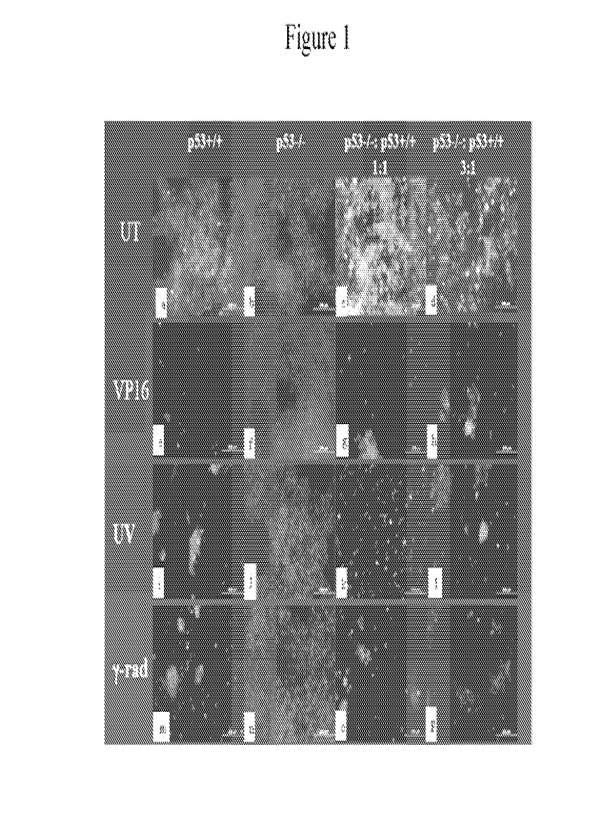

This experimental system mimics restoration of p53 function of a p53-deficient cell population as would occur in p53 gene therapy. To achieve this parental wt-p53 HCT116 colon carcinoma cells were mixed with isogenic p53 knockout HCT116 cells at 1:1 ratio and examined whether p53 activation by chemical DNA damage would selectively kill the wt-p53 cells or also result in bystander killing of the p53 null cells. To distinguish the two cell populations, the cells were labeled with two different fluorescent dyes; parental wt-p53 positive cells in green and p53-null cells in red. The cells were diffusely stained and could be reliably tracked for at least 72 h, after which the fluorescence declines due to cell division. Etoposide treatment of the mixed cell populations demonstrated uniform induction of cell death after 24 h as demonstrated by reduced number of cells visible per field, while when kept separate only the wt-p53 cells died (FIG. 1), indicating a soluble factor secreted by the wt-p53 expressing cells in response to p53 activation by etoposide might underlie this effect.

To determine whether other p53 activation stimuli could elicit similar paracrine cell death induction, wt-p53 and p53 knockout HCT116 cells were treated with ultraviolet (UV) and gamma-irradiation and repeated the treatments with etoposide. The mixed experiments to test the influence of wt-p53 cells on p53-null cells at cell ratios of 1:1 and 1:3 etoposide, UV and gamma-irradiation treatment of the mixed cell populations at 1:1 and 1:3 ratios demonstrated uniform induction of cell death after 24 h as demonstrated by reduced number of cells visible per field, while when kept separate only the wt-p53 cells died (FIG. 1), indicating a soluble factor secreted by the wt-p53 expressing cells in response to p53 activation by varied genotoxic stimuli (etoposide, UV and gamma-irradiation) might underlie this effect.

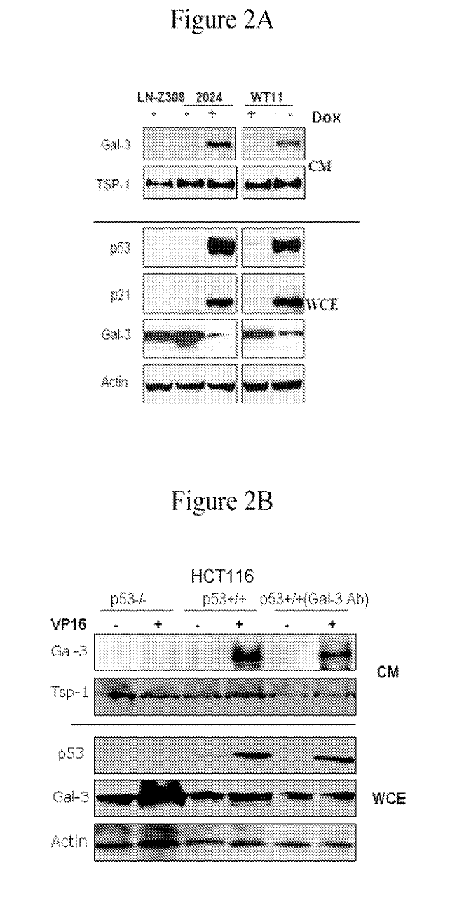

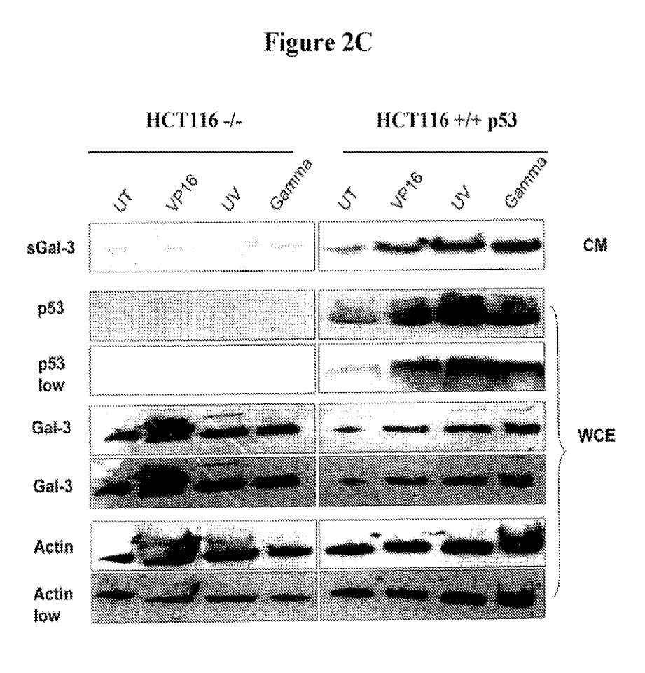

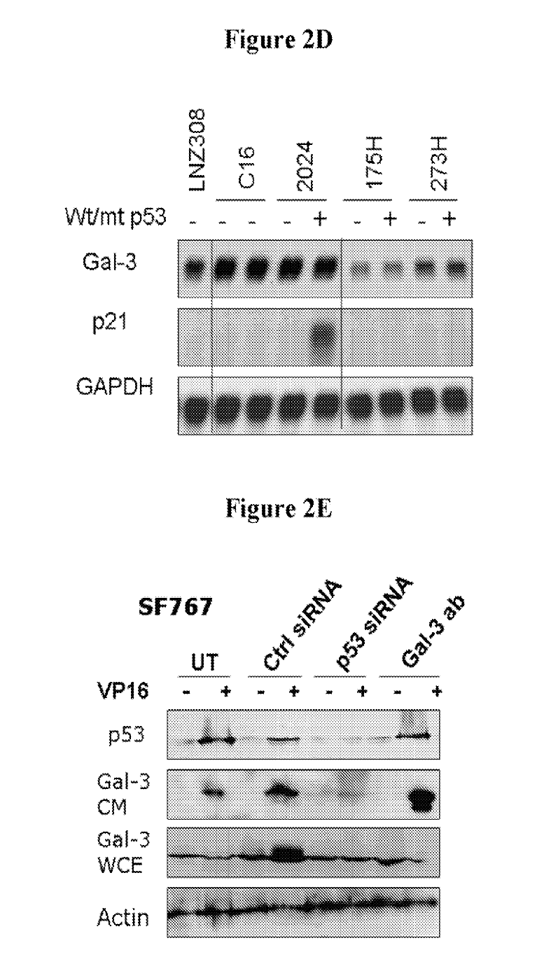

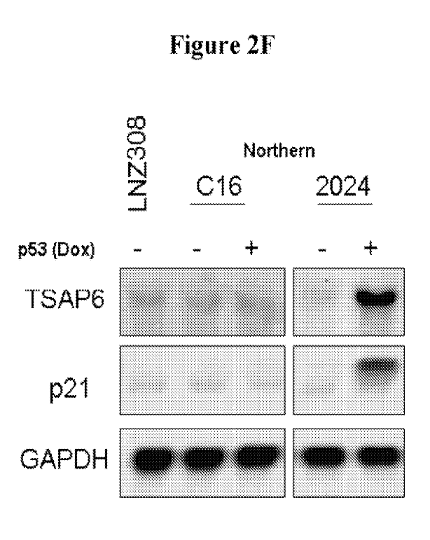

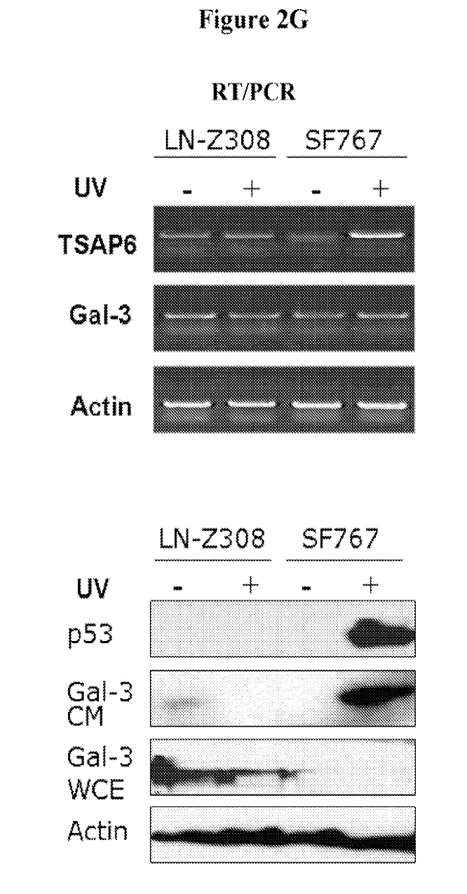

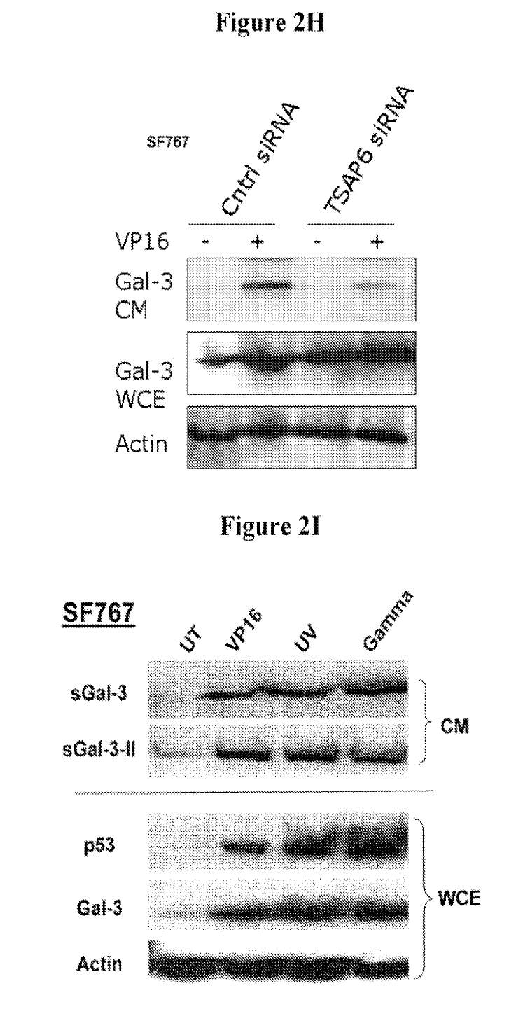

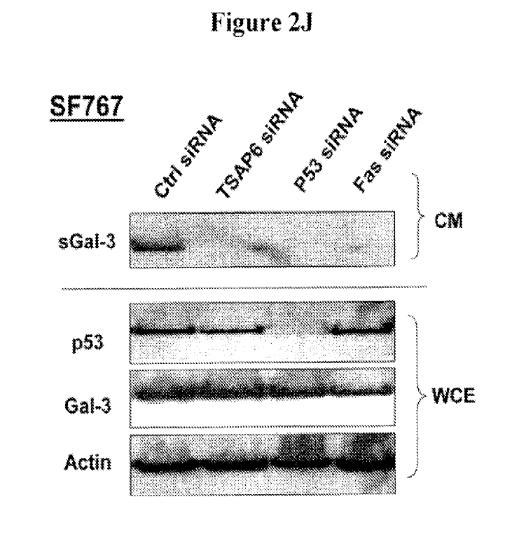

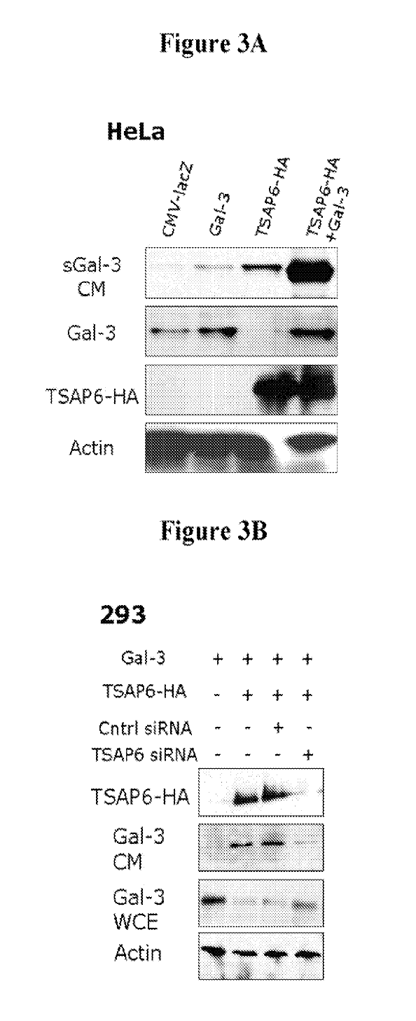

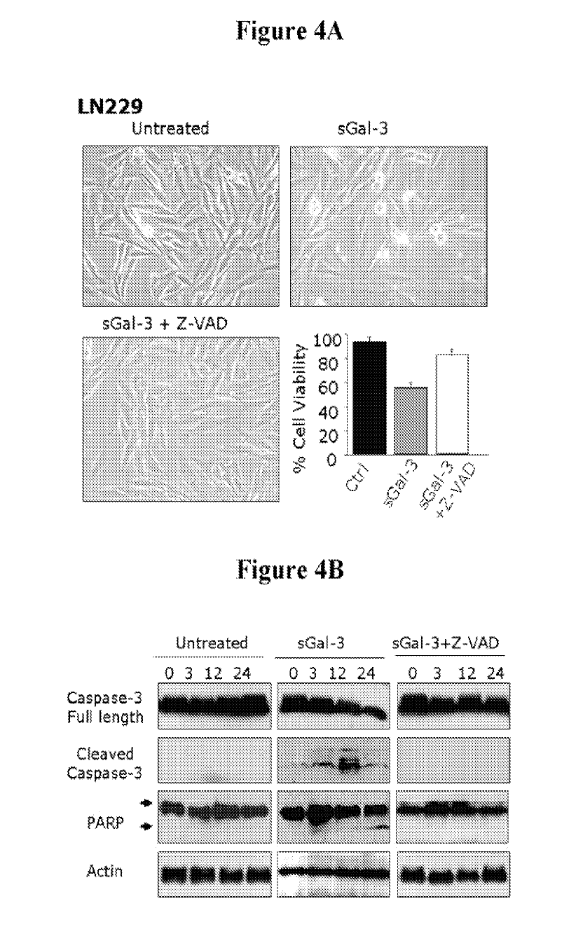

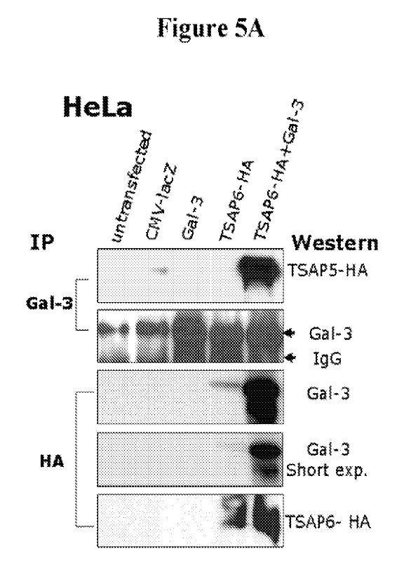

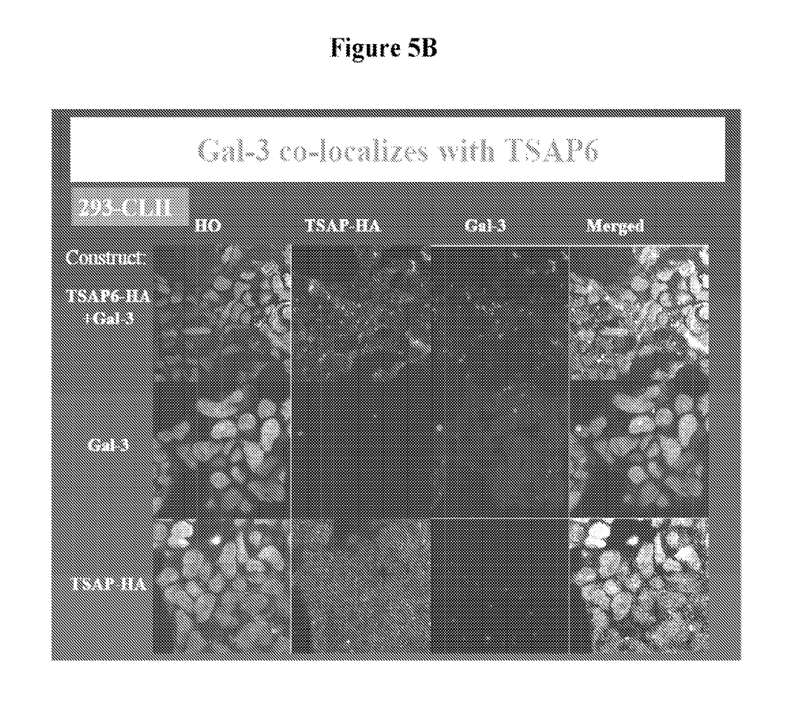

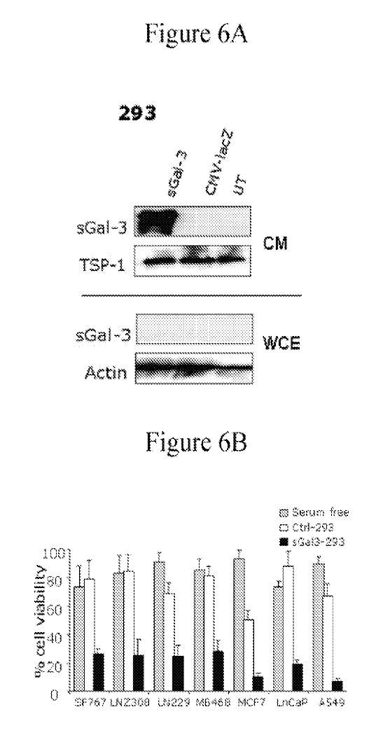

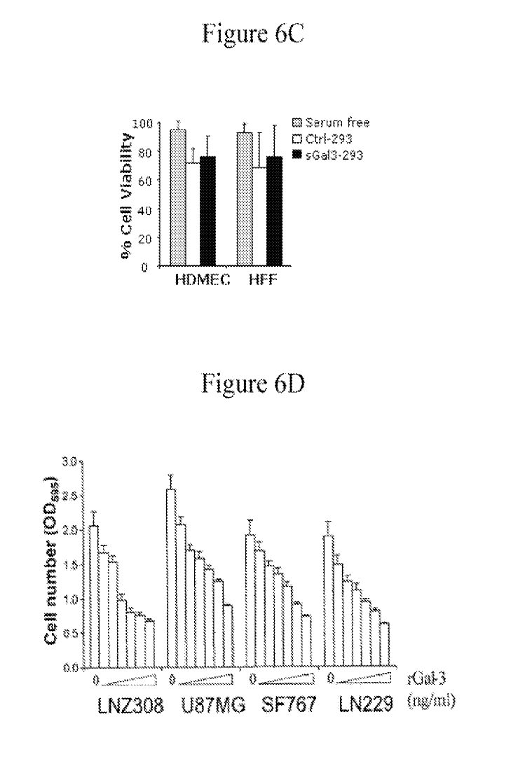

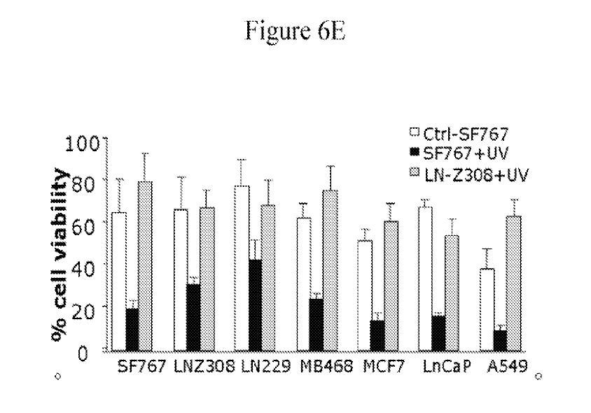

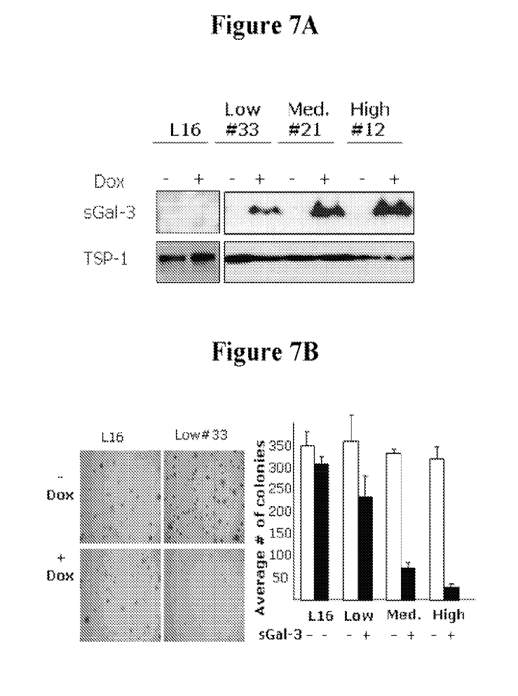

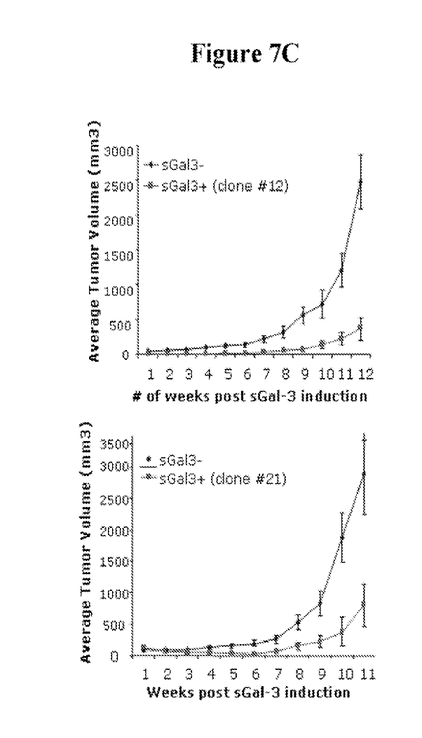

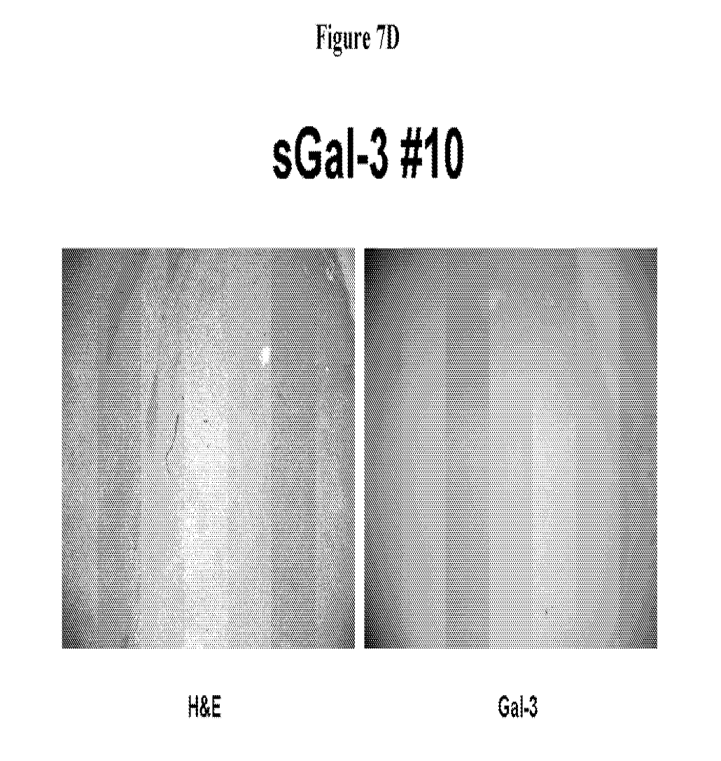

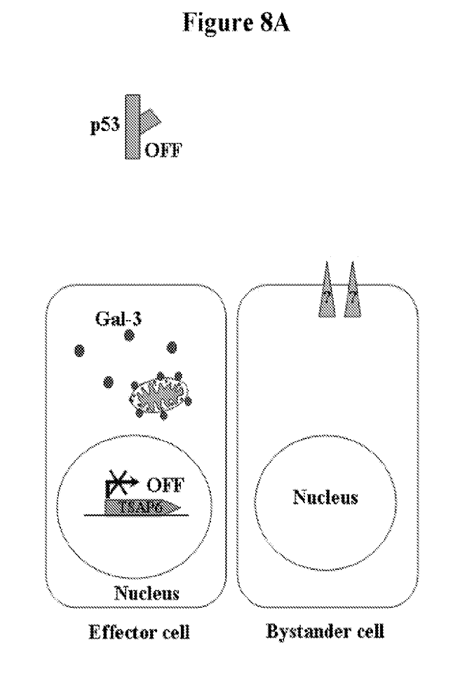

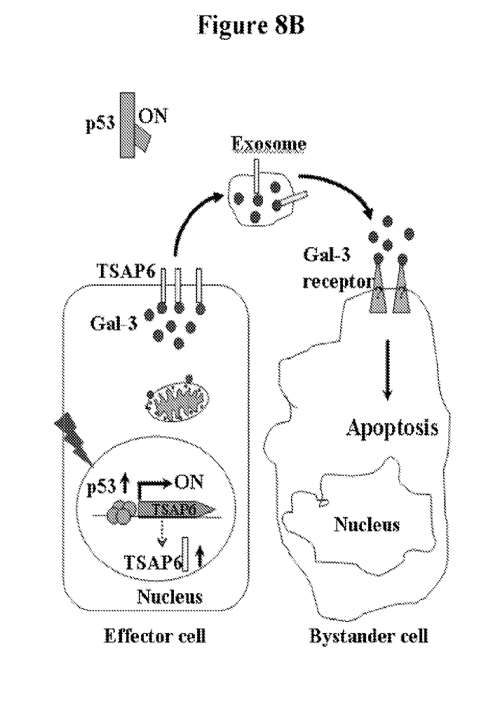

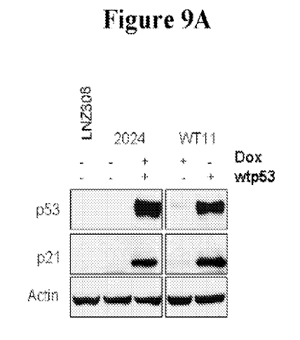

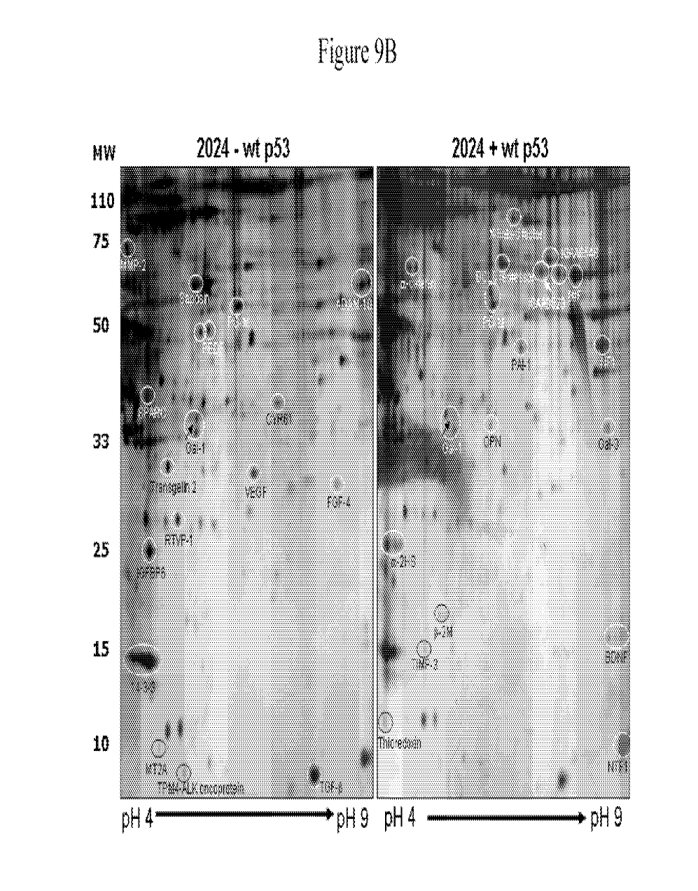

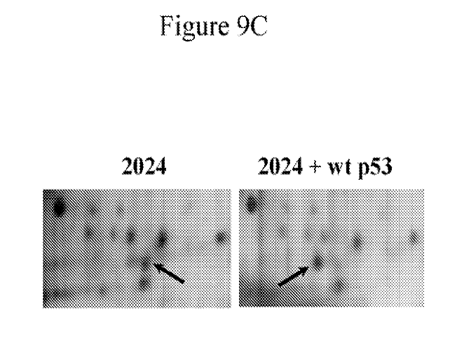

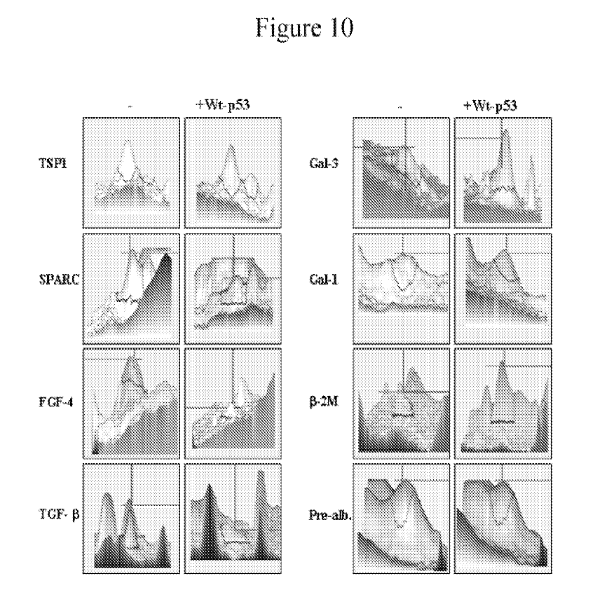

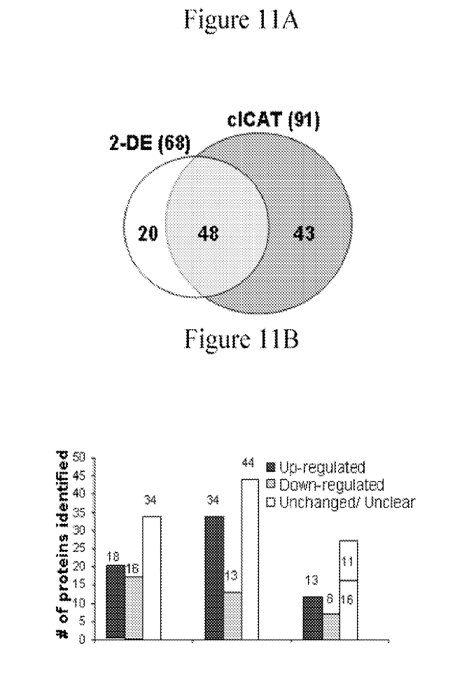

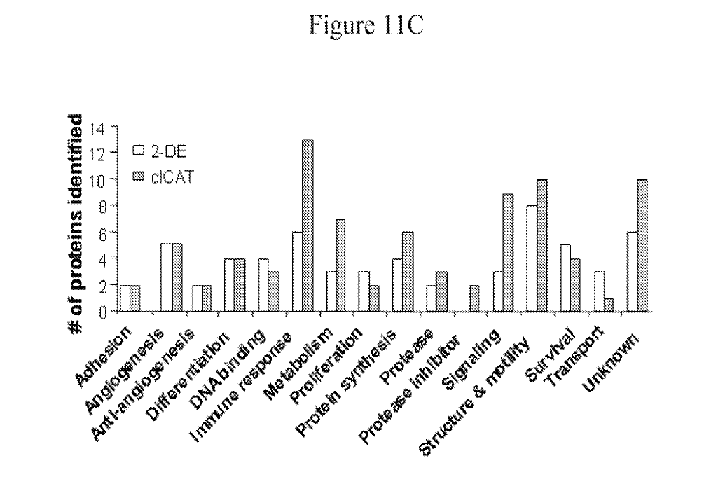

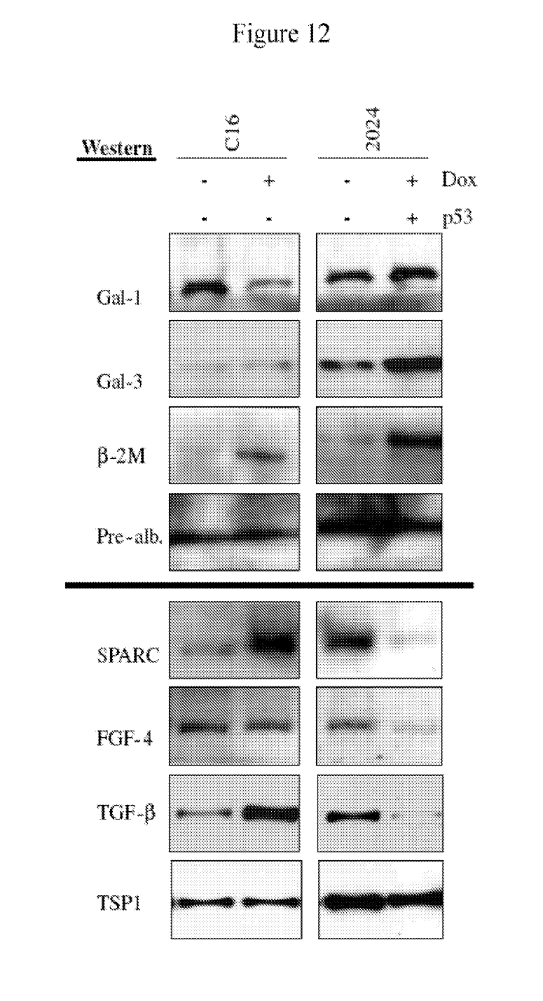

Supernatant from wt-p53 HCT116 colon cancer cells +/−UV treatment was further collected and used on p53 knockout cells to examine cell killing. UV activation of p53 was used to avoid the presence of etoposide in the conditioned media (CM). Conditioned media (CM) from UV treated wt-p53 cells induced cell death in p53 knockout cells while CM from non-treated cells or UV treated p53 knockout cells had no effect (FIG. 2C). These results suggest that p53 activation by UV or chemotherapy can induce killing of bystander cells through the release of a soluble death-inducing factor.

Gal-3 Secretion is Enhanced by wt-p53 and is Necessary or the p53Bystander Effect:

To identify potential extracellular regulators of cell death under the control of p53, the secreted proteome of a p53-null glioma cell line (2024; p53 tet-on) was queried with a dox-inducible wt-p53 (Khwaja et. al. 2006). Secreted proteins were separated by two-dimensional gel electrophoresis (2-DE) and comparative analysis using the ImageMaster software revealed a 2-3 fold increase in secreted levels of pro-apoptotic factor Gal-3 upon wt-p53 induction (FIG. 10). This was confirmed by western analysis in 2024 cells and in a second p53 inducible cell line (WT11, p53 tet-off). In both clones, robust induction of wt-p53 expression is seen 48 h post induction along with an increase in the p21 cell cycle inhibitor, a well-known p53 target (FIG. 2A). Both cell lines displayed increased release of Gal-3 in the CM with a concomitant decrease in its intracellular levels (FIG. 2A). Thrombospondin-1 (TSP-1) secretion is not induced by p53 in our experimental system (Tenan et al. 2000) and was used a negative control.

Gal-3 secretion was enhanced upon induction of p53 in the HCT116 colon carcinoma cell lines used in our mixing experiments as well (FIGS. 2B, 2E). To determine whether p53 activating genotoxic treatments (VP16, UV and gamma-irradiation) influenced Gal3 secreted Gal-3 levels, western blots on CM and whole cell extracts (WCE) of HCT116 cells were performed (wt-p53 or p53-null). Western blot showed extracellular accumulation (CM) of Gal-3 in response to p53 stabilization by VP16 (25 uM), UV (60 mJ per cm²) and gamma-irradiation (10 Gy) in p53^{+/+} but not in p53-null HCT116 cells 18 hrs post treatment.

The intracellular levels of Gal3, p53 and actin in the whole cell extract (WCE) were also examined. The genotoxic treatments stabilized p53 cellular levels only in p53^{+/+} cells as expected. This observed increased levels of Gal3 in the CM of p53^{+/+} cells but not in p53^{-/-} cells, demonstrates that p53 is necessary to mediate this effect. Importantly, anti-Gal-3 antibody was able to prevent the bystander cell killing in the cell mixing experiments and neutralized the cell death induced by CM from UV treated wt-p53 expressing HCT116 cells (FIG. 2C). As a control it was verified that the presence of Gal-3 antibody in the media did not preclude activation of p53 and Gal-3 secretion (FIG. 2B). Similar results were obtained in SF767 human glioma cells, with all three genotoxic treatments activating p53 intracellular p53 stabilization, and extracellular accumulation of Gal-3 (FIG. 2I). These results suggest that p53-mediated secretion of Gal-3 is an important contributor of the p53 bystander effect.

The LGALS3 Gene is not a Direct Target of wt-p53:

To investigate the mechanism by which p53 can regulate Gal-3 extracellular levels it was first examined whether LGALS-3 was a direct target gene of p53. The levels of Gal-3 transcripts were examined by Northern blot in isogenic clones of the LN-Z308 (p53 null) cell line with inducible Wt-p53 (2024) or mutant p53 (175H and 273H) as controls (FIG. 2D). Transcriptional activation of the CDKN1 gene by wt-p53 was used as a positive control. Gal-3 mRNA levels remained constant regardless of p53 status in the cells, suggesting that LGALS3 gene transcription was not under p53 control. Modulation of endogenous wt-p53 by UV in SF767 cells did not modify Gal-3 mRNA expression either (FIG. 2G).

p53 Activation of TSAP6 Expression Correlates with Enhanced Secretion of Gal-3:

Since Gal-3 was not a direct target of p53, it suggests that p53 may influence its secretion. To find a possible mediator of the p53 effects on Gal-3 secretion, TSAP6 (tumor suppressor activated pathway-6), a 6-transmembrane protein recently reported to facilitate the secretion of proteins by ectocytosis (Amzallag et al. 2004; Yu et al. 2006), was tested to determined whether TSAP6 is the mediator of p53 regulated Gal-3 secretion.

First, whether TSAP6 was expressed and regulated by p53 in the human glioma cell lines were tested. Northern blot analysis showed strong upregulation of the TSAP6 transcript levels by wt-p53 expression in 2024 cells (FIG. 2F). LNZ308-C16 parental cells (Albertoni et al. 1998) were used as a control for the tet-inducible cell lines to ensure that dox treatment does not affect TSAP6 or Gal-3 levels (FIG. 2F). To further confirm that TSAP6 upregulation could occur under physiological activation of endogenous wt-p53, the SF767 cell line, a wild-type cell line for p53 was used. In these cells, a marked up-regulation of the TSAP6 transcript levels was observed upon p53 activation by DNA damage induced by UV treatment. No increase was seen after similar treatment in p53-null LN-Z308 cells (FIG. 2G, upper panel).

Next, whether the UV-induced upregulation of endogenous TSAP6 in SF767 cells (FIG. 2G, upper panel) resulted in concomitant enhanced secretion of Gal-3 was tested. A marked increase in secreted Gal-3 was seen upon activation of p53 following UV treatment (FIG. 2G, lower panel).

To further examine whether p53 activation through chemotherapy would have similar effects SF767 cells were treated with either etoposide (VP16) alone or in the presence of either control or p53 specific siRNAs. Activation of p53 by VP16 treatment led to enhanced secretion of Gal-3 in the media. This effect was inhibited in the presence of siRNAs for p53 but not control siRNAs (FIG. 2H).

To extend these observations to UV treatment SF767 cells were treated with UV and observed accumulation of extracellular Gal-3. This effect was inhibited by siRNAs against TSAP6, p53 and Fas (FIG. 2J). Intracellular levels of Gal3 and actin were unaffected. Altogether these data show that p53 activation by UV or chemotherapy induces TSAP6 expression and Gal-3 secretion.

### Wt-p53 Regulated TSAP6 is Necessary for Gal-3 Secretion:

To evaluate whether there was a causal relationship between TSAP6 expression and Gal-3 secretion, the effects of overexpressing HA-tagged TSAP6 on the localization of endogenous and extracellular Gal-3 were investigated. HeLa cells were transfected with either a pCMV-lacZ control plasmid or with expression vectors for Gal-3 or TSAP6-HA. The cells were placed in serum-free medium and CM was collected 48 h later and assayed for the presence of secreted Gal-3. Gal-3 was almost undetectable in the CM of pCMV-lacZ transfected cells (FIG. 3A, lane 1). Transfection of a Gal-3 expression plasmid led to a substantial increase in intracellular and secreted Gal-3 (lane 2). Substantially higher levels of Gal-3 were released in the CM after transfection of HA-tagged TSAP6 (lane 3) indicating that endogenous Gal-3 stores were relocated to the extracellular milieu in response to TSAP6 overexpression. Co-transfection of Gal-3 and TSAP6-HA expression vectors resulted in maximal Gal-3 secretion (lane 4). Immunoblotting of cellular extracts with α-Gal-3 and α-HA antibodies confirmed expression of the transfected proteins. These results indicate that increased cellular TSAP6 levels facilitate the secretion of Gal-3 and deplete intracellular stores of Gal-3.

To examine whether a reduction in TSAP6 levels abrogates Gal-3 secretion, siRNA was used against TSAP6 in Gal-3 transfected HEK293 cells (with undetectable endogenous Gal-3). The cells were co-transfected with either HA-tagged TSAP6 expression vector alone or in combination with control or TSAP6-specific siRNA. Gal-3 transfection alone did not result in detectable levels of secreted Gal-3 (FIG. 3B; lane 1). Cotransfection with TSAP6-HA clearly induced the secretion of Gal-3 in the CM with a concomitant decrease in intracellular Gal-3 levels (lane 2). Transfection with TSAP6-specific siRNA eliminated this effect (lane 4). Inhibition of endogenous TSAP6 expression by siRNA also reduced Gal-3 secretion in SF767 cells (FIG. 2E). These results support the conclusion that TSAP6 is necessary for the extracellular secretion of Gal-3.

Gal-3 Interacts with TSAP6:

To examine whether Gal-3 might interact with TSAP6, co-immunoprecipitation assays were performed on HeLa cells transfected as above and immunoprecipitated Gal-3 and TSAP6-HA with either α-Gal-3 or α-HA antibodies followed by Western blot analysis (FIG. 5A). In cells co-transfected with both Gal-3 and TSAP6-HA expression vectors, an association between TSAP6 and Gal-3 was found. Immunoprecipitation with α-Gal-3 pulled down transfected TSAP6-HA (FIG. 5A; Blot 1; lane 5) and conversely, immunoprecipitation with α-HA pulled down both endogenous and transfected Gal-3 (FIG. 5A; Blot 3; lanes 4 & 5). These results provide evidence for a physical association between Gal3 and TSAP6, either directly or in a complex.

To further confirm that a Gal-3 interaction with TSAP6 is present in vivo, we examined whether the two proteins co-localized by confocal microscopy. HEK293 and HeLa cells were transfected with expression vectors for Gal-3 and HA-tagged TSAP6 singly or in combination, and a co-immunofluorescence experiment was performed (FIG. 5B). TSAP6 expression was seen mostly in the cell membrane as expected, but also in vesicles within the cells (FIG. 5B; green). Gal-3 expression was found to be largely cytoplasmic with the majority of the Gal-3 protein contained in aggregates or vesicles (FIG. 5B; red). In co-transfected cells, the Gal-3 and TSAP6 signals were found to co-localize in intracellular aggregates as well as in parts of the membrane (FIG. 5B; yellow). These observations support the conclusion that TSAP6 interacts with Gal-3 at the cell membrane and in intracellular vesicles.

### Secreted Galectin-3 Reduces Glioma Cell Viability In Vitro:

In a preliminary experiment, it was found that the addition of increasing amounts of recombinant Gal-3 (rGal-3) in the culture medium led to a dose-dependent decrease in cell number over 48 h in four different human glioma cell lines (FIG. 6D). These results suggested a cytotoxic effect of secreted Gal-3 on glioma cells. It is noted that the potency of the recombinant Gal3 is less than that found naturally secreted by human cells, perhaps because of a lack of post-translational modifications. A comparison of rGal3 with naturally secreted Gal3 evidences post-translational modifications in the naturally secreted one (data not shown).

To determine the effect of augmented extracellular Gal-3 levels without the confounding effects of altering the endogenous levels of TSAP6 and other elements in the non-classical secretion pathway, an expression vector able to mediate rapid secretion of Gal-3 in a variety of cell lines was constructed. To avoid potential artifacts due to intracellular accumulation of Gal-3, the Gal-3 cDNA was fused to a classical secretion signal peptide sequence from the plasminogen activator gene, and a vector expressing constitutively secreted soluble Gal-3 (sGal-3) was generated.

After cleavage of the secretion signal, the sGal-3 amino acid sequence is identical to the endogenous secretion product. It is nevertheless possible that sGal3 will have different post-translational modifications that may vary between the exosome-mediated secretory pathway and the classical secretion pathway. To verify the functionality of the construct, the construct was transfected in 293-CLH cells where it mediated a robust increase in the expression and secretion of Gal-3 without intracellular accumulation (FIG. 6A). Using the CM of transfected 293 cells as a source of sGal-3, its effect on the growth of SF767, LN-Z308 and LN229 glioma cells was determined using a crystal violet assay. All three cell lines exhibited a decrease greater than 75% in cell number after 48 h. Cells incubated with serum free medium or CM from 293 cells transfected with control pCMV-lacZ plasmid were unaffected (FIG. 6B).

To examine whether the cytotoxic effect of Gal-3 was limited to glioma or whether it represents a more general anti-cancer property, two breast cancer (MD468 and MCF7), two lung cancer (A549 and H1289) and two prostate cancer (LnCaP and PC3) cell lines were tested using the same assay and found all of them to be sensitive to sGal-3 presence in the medium (FIG. 6B). In contrast, cultures of normal fibroblasts or endothelial cells (HFF and HDMEC) did not show decreased viability in response to secreted Gal-3 in the media (FIG. 6C). Taken together, these results show that CM from cells overexpressing sGal-3 displays a tumor-cell specific cytotoxicity.

To determine whether this cytotoxic effect occurs with physiological levels of Gal-3 secreted in response to the activation of endogenous wt-p53, the same panel of cancer cell lines were treated with media collected from p53-null LNZ308 or wt-p53 SF767 glioma cells after UV treatment to activate p53 mediated Gal-3 secretion. Cytotoxic effects were observed in all tested cancer cells only when CM from UV-treated SF767 cells was used (FIG. 6E). CM from UV treated LN-Z308 or untreated SF767 cells had no effect. These results suggest that the physiological levels of Gal-3 secreted in response to p53 activation are sufficient to mediate cytotoxic effects on a variety of cancer cells of epithelial and neuroectodermal origin.

### Secreted Gal-3 Induces Apoptosis to Glioma Cells:

Since extracellular Gal-3 has been implicated in the promotion of T-cell apoptosis (Fukumori et al. 2003), its cytotoxic effects linked to induction of programmed cell death in cancer cells were tested. LN229 and LN-Z308 glioma cells were treated with either extracellular Gal-3 alone, or in combination with the Z-VAD general caspase inhibitor and analyzed the cells for apoptotic features. Cells treated with sGal-3 alone showed membrane blebbing, and cell death as early as 12 h post treatment. This effect was inhibited in the presence of Z-VAD indicating that the observed cell death was caspase-mediated, thereby suggesting an apoptotic response (FIG. 4A). This contention was validated by the observation of Caspase-3 and PARP cleavage in the cells within 12 h of sGal-3 treatment, an effect neutralized by Z-VAD (FIG. 4B). These data demonstrate that treatment of cancer cells with sGal-3 promotes apoptosis.

The levels of cell death in SF767 cells in response to VP16 treatment in the presence of either p53 or TSAP-6 specific siRNAs as well as in the presence of anti-Gal-3 antibody were further determined. VP16 treatment alone or in the presence of control siRNA led to marked levels of apoptosis. This effect was inhibited in the presence of specific siRNAs for p53 (FIGS. 2E and H) or TSAP-6 as well as in the presence of anti-Gal-3 antibody. The inhibition mediated by TSAP6 siRNA was not complete, likely reflecting the incomplete TSAP6 inhibition by this siRNA (FIG. 2E). These results strongly implicate secreted Gal-3 in p53-induced paracrine apoptosis of cancer cells.

### Secreted Gal-3 Reduces Tumorigenicity of Glioma Cells In Vitro and In Vivo:

To determine that p53-induced sGal-3-mediated apoptosis may serve as one of the tumor suppressive mechanisms that exerts selective pressure for the survival and clonal expansion of cells with TP53 gene mutations during the process of tumor development, it is suggested that forced sGal-3 secretion by tumor cells would reduce tumor growth. To test this concept, dox-inducible sGal-3 clones were generated with variable expression levels in LN229-L16 cells (rtTA expressing) (see materials and methods) (FIG. 7A). Induction of sGal-3 led to cell death in all three clones within 3-5 days in cell culture while LN229-L16 parental cells were unaffected (data not shown).

To examine whether cell death would also be observed in anchorage-independent growth conditions, more reminiscent of in vivo growth, soft-agar colony formation assays were performed. The sGal-3 induction resulted in reduced colony formation in a dose-dependent manner with a 2- to 6-fold decrease in the number of colonies formed, proportional to increasing amounts of sGal-3 produced in the CM (FIG. 7B). These results confirm that secreted Gal-3 inhibits anchorage-independent growth, an essential step for tumor formation.

To test directly the effect of sGal-3 on tumor growth in vivo, a nu/nu mouse xenograft model system was used. Cells (5×10⁶) from the sGal-3 expressing tet-inducible clones #12 and #21 were injected subcutaneously in the mice and sGal-3 expression was induced one week post tumor cell implantation in half of the injected mice by feeding dox in the drinking water for 12 weeks (9 mice/group). Only 4 of the 18 tumors (clone #12) in the sGal-3 expressing group developed while 14/18 tumors grew in the control group. Moreover, the sGal-3 expressing tumors developed much slower than the control group, with average tumor volumes at termination over six-fold smaller than the controls (p<0.02) (FIG. 7C). Similar results were seen with clone #21 as well where only 6 of 18 tumors grew in the presence of sGal-3 expression compared to 16/18 control tumors and tumors expressing sGal-3 were on average 4 fold smaller at termination than in the control group (FIG. 7C). Immunohistochemistry for Gal-3 expression in excised tumors from mice treated with doxycycline confirmed that Gal-3 was expressed (FIG. 7D).

In summary, these data demonstrate that secreted Gal-3 potently inhibits tumor formation and growth in vivo.

### Example 2

### Proteomic Identification of the wt-p53-Regulated Tumor Cell Secretome

### Materials and Methods:

### Cell Lines and Culturing Conditions:

LN-Z308 (p53 null) human glioblastoma cell line (Albertoni et al. 1998), and its isogenic clones LNZ-308-C16 (contains a reverse tetracycline transactivator (rtTA)), 2024 (tet-inducible wt-p53) (Albertoni et al. 2002) and WT11 (tet-off for wt-p53) (Van Meir et al. 1994) were grown in DMEM supplemented with 5% FCS. Cells were grown in serum-free media and wt-p53 expression was induced by modulation with 2 μg/ml of doxycycline (dox). Conditioned media (CM) from the cells was collected after 48 h induction and frozen at −20° C. after removal of floating cells and cell debris by centrifugation at 1,000 g.

### Two-Dimensional Polyacrylamide Gel Electrophoresis (2-De):

Samples were analyzed in triplicates using 2-DE as described (Goldman et al. 1980). The first dimension was performed on IPGphor system (Amersham Biosciences, NJ, USA). Isoelectric focusing of 200 μg of TCA precipitated protein was performed on 13 cm or 17 cm Immobiline dry strips (IPG strips) using either pH range of 3-10NL or 4-7L, (total run=130,000 Vh). Strips were then equilibrated sequentially in equilibration buffer (6M urea, 2% SDS, 0.05M tris base pH 8.8, 20% glycerol) first containing 10 mg/ml DTT and then 25 mg/ml iodoacetamide followed by separation in the second dimension on 12.5% polyacrylamide gels with 2% SDS using the Protean II xi system (BioRad, CA, USA). Silver Stain Plus kit (BioRad) was used to visualize protein spots and the gels were analyzed using Melanie, and the ImageMaster softwares (Amersham Biosciences, NJ, USA).

### In-Gel Digestion of Proteins and MALDI-TOF/TOF-Ms Analysis:

Protein spots of interest were excised from the gel and destained using SilverOUT kit (GenoTech, MO, USA). The proteins were digested overnight with 150 ng trypsin (Promega, WI, USA) and the resulting peptides extracted using Montage In-gel peptide extraction kit (Millipore, MA, USA), spotted onto target plates and overlaid with alpha-cyanocinnaminic acid matrix (Agilent, DE, USA). The plates were analyzed using a 4700 Proteomics Analyzer (Applied Biosystems, CA, USA). The combined MS and MS/MS spectra from each spot were processed using GPS Explorer V2.0 (Applied Biosystems, CA, USA) with MASCOT (Matrix Science, MA, USA) as the database search engine. Only proteins that generated multiple peptides with ion scores above 30 were considered positively identified.

Cleavable Isotope-Coded Affinity Tag (cICAT) Analysis:

cICAT technology uses stable isotope tags in combination with two-dimensional chromatography of complex peptide mixtures (Applied Biosystems, CA, USA) (Gygi et al. 2002). 100 μg each of precipitated secreted protein from the CM were treated with denaturing (50 mM Tris; 0.1% SDS) and reducing (50 mM TCEP (Tris(2-carboxyethyl)phosphine hydrochloride)) reagents. Next, the control and wt-p53 induced samples were respectively labeled with light (9 ¹²C atoms) and heavy (9 ¹³C atoms) reagents for 2 h at 37° C. After trypsin digestion and purification, the peptides were analyzed using an Ultimate nanoHPLC LC-MS/MS (Dionex/LC Packings, CA, USA) interfaced to a QSTAR XL mass spectrometer (Applied Biosystems, CA, USA). The MS/MS data was processed using ProICAT software for protein identification and quantification. Only proteins with ProtScore>1.0 (>85% confidence) were considered. Also, the heavy to light ratios were tested for significance using student t-test and p<0.05 was considered significant.

### Western Blot Analysis:

Immunoblots were performed on cell lysates (lysed in 8 M urea, 4% SDS, in 10 mM Tris (pH 7.4)). The conditioned media was precipitated by 15% TCA-precipitation for 2 h at 4° C., washed twice with ice cold acetone, and then resuspended in lysis buffer (8M urea, 4% SDS, 100 mM protease inhibitor cocktail (Roche), in 10 mM Tris (pH 7.4)). Antibodies used were: α-TSP1 (Ab-4 NeoMarkers, Freemont, Calif.; 1:1000), α-FGF-4 (sc-16812, Santa Cruz, Calif., USA; 1:500), α-SPARC (sc-13324, Santa Cruz; 1:500), α-VEGF (Santa Cruz; 1:500), α-β2-microglobulin (Clone B2M-01; Abeam, Mass., USA; 1:250), α-TGFβ (AE1109.1, Immunodiagnostik, Germany; 1:100), α-galectin-3 (Santa Cruz, Calif., USA; sc-14364; 1:500), α-galectin-1 (Santa Cruz, Calif.; 1:500). Pre-albumin was (se-13098; Santa Cruz; 1:1000) and actin (sc-1615; Santa Cruz, Calif.; 1:1000) were used as a loading control

### Results:

To identify p53-regulated extracellular proteins involved in the cell-cell communication events important for human cell transformation, the LN-Z308 cell line was collected as it derived from a malignant human glioma that lost both p53 alleles in vivo by well-characterized genetic events suggesting selective pressure for their loss. Reactivating wt-p53 function in these cells reverts or restores the release of p53-regulated secreted proteins and allows their identification in the conditioned media (CM) (Albertoniet al. 1998). Isogenic clones of LN-Z308 with tetracycline-inducible (2024) (Albertoni et al. 2002) and repressable (WT11) (Van Meir et al. 1994) wt-p53 expression were used (FIG. 9). These cells undergo growth arrest but not apoptosis in response to p53 (Van Meir et al. 1994) and show induction of the cell cycle inhibitor p21 upon p53 induction (FIG. 9). Using this system, differential profiles of the cell lines' secretome with and without wt-p53 expression were generated using two complementary proteomic techniques: 2-DE and cICAT.

Two-Dimensional Gel Electrophoresis (2-DE) of the Tumor Cell Secretome:

Extracellular proteins were separated by 2-DE analysis using non-linear pH range of 3-10 and linear range of 4-7 in triplicates to ensure reproducibility (Gorg et al. 2004). The proteins were visualized by silver staining and analyzed using ImageMaster software. As a further precaution against artifacts, both 2024 and WT11 clones were profiled and only proteins found secreted in both were retained.

The protein spots were next excised from the gel, subjected to in-gel digestion with trypsin and identified using MALDI-TOF/TOF MS analysis (Tables 1 and 3). It was found on average >150 spots on each gel representing 68 individual proteins (FIG. 10; Tables 1-3). A semi-quantitative analysis of this differential expression was partially quantified by comparing spot intensity and volume using ImageMaster (FIG. 11). The levels of 34 proteins in the CM were found to be largely invariable regardless of p53 expression, whereas 32 individual proteins showed differential expression levels in the CM in response to p53 (Tables 1-3). Among the differentially expressed proteins, 18 had increased levels and 16 decreased levels in the CM in response to wt-p53 expression in the cells (FIG. 12B). The 68 secreted proteins identified in the 2-DE screen belonged to 15 functional categories (FIG. 12C; Tables 1 & 2).

Secretome Analysis by Cleavable Isotope-Coded Affinity Tag (cICAT): Recently, internally standardized gel-free quantitative proteomic methods have been developed to alleviate limitations of 2-DE. One of these methods is isotope-coded affinity tag (ICAT) reagent labeling and tandem mass spectrometry (MS/MS) (Gygi et al. 2002). Secreted proteins from 2024 and WT11 cells were very similar in their expression patterns and differed significantly in expression levels for only 10 of the 91 proteins identified by this analysis. Through cICAT alone, 34 proteins were found with increased levels, and 13 with decreased levels by at least 20% while 43 remained unchanged in response to wt-p53 expression (Tables 1-3; FIG. 12B). These proteins were found differentially expressed in CM (p<0.05) in at least 2 of the 3 experiments for both cell lines. The quantification from cICAT was found to be consistent between experiments as seen by small standard deviation values for each tested cell line (Tables 1-3). Similar to 2-DE results, the 91 proteins found secreted in the media by cICAT experiments belonged to 15 functional groups (FIG. 12C; Tables 1-2).

Comparison of 2-DE and cICAT Results:

Combining both techniques, 111 separate extracellular proteins were identified; 68 by 2-DE and 91 by cICAT analysis (FIG. 12A). It is noteworthy that 48 of the 91 (˜50%) secreted proteins identified by cICAT analysis, were identical to the ones identified by 2-DE analysis, showing concordant results between the techniques in identifying the complement of secreted proteins (Tables 1-3; FIG. 12). 37 of the 48 (77%) proteins commonly identified by the techniques showed similar responses to p53 activation in the cells. The majority of the remaining 11 (24%) proteins (listed as U) were either secreted at a very low level or not differentially expressed to a high degree. When looking at the concordance between p53 regulated proteins, 13 proteins were found up-regulated, 8 proteins were found down-regulated and 16 proteins were found unchanged by both 2-DE and cICAT analysis while the remaining 11 (listed as U) were found differentially expressed only by one of the two indicated methods (FIG. 12B; Tables 1-3).

### Verification of Proteomic Results:

Some of the p53-regulated secreted proteins found in the analysis had been previously reported and validated, including VEGF (Miyagami and Katayama 2005), osteopontin (Morimoto et al. 2002), SPARC and dickkopf (Wang et al. 2000). To confirm the results three proteins whose levels were increased (galectin-1, galectin-3 and β-2M) and three decreased (SPARC. FGF-4 and TGF-β) in the CM in response to wt-p53 expression were picked for validation by western analysis. The levels of Gal-1, Gal-3 and β-2M in the CM were clearly increased by p53 in 2024 cells (FIG. 12, compare lanes 2 and 4). In contrast, secreted levels of SPARC, TGF-β, and FGF-4 were decreased. The downregulation of SPARC and TGF-β levels in the CM by p53 was particularly strong as it was able to antagonize their increase by doxycycline as seen in the C16 control cells that lack p53. TSP-1 was used as a loading control since its levels are not found to be increased by wt-p53 in our glioma system (Tenan et al. 2000). The data show that our proteomic analysis with 2-DE and cICAT can be used to reliably identify differential expression of secreted proteins in the CM (Tables 1-3).

### Investigation of the Mechanism Underlying p53 Control Over Protein Secretion:

To examine whether the CM levels of the extracellular proteins identified were regulated by p53 at the gene expression level, the differential expression of their mRNAs by microarrays in the 2024 cell line was examined in three independent experiments. None of the mRNAs corresponding to the secreted products found in the analysis appeared to have levels significantly modulated by p53 (Table 1; column 5). These findings suggest a role of wt-p53 in the modulation of the extracellular levels of secreted proteins through either enhanced stability or secretion. One way that p53 could potentially affect protein stability and/or secretion is through regulation of post-translational modifications e.g. phosphorylation, glycosylation acetylation and hydroxylation of proteins, events that may mark certain proteins either for degradation or for localization (Kamemura and Hart 2003).

Preliminary indications of such post-transcriptional modifications were noted for a subset of the identified proteins through 2-DE analysis (Table 4), as seen for example by the horizontal and vertical shifts of Gal-1 protein spots from their original pI and MW positions (FIG. 10, Black arrow). This suggests a potential novel function of the p53 tumor suppressor, the modulation of post-transcriptional modifications. Alternatively, p53 may also be involved in the regulation of a specific secretory pathway (Yu et al., 2006). Indeed, most proteins whose levels were positively regulated by p53 were found secreted through non-classical mechanisms including vesicle-mediated pathways like exocytosis, ectocytosis as well as through transporter-mediated pathways (Tables 1-2). In contrast, most proteins released through classical pathways were down-regulated (Tables 1-2).

TABLE 1
2024 WT11

| Protein name | Acces. # | MW | pI | mRNA | 2-DE | H:L | st. Dev | H:L | st. Dev |
|---|---|---|---|---|---|---|---|---|---|
| Galectin-3 | gi:4504983 | 26.19 | 8.58 | 1.05 | C | 1.60 | 0.12 | 1.53 | 0.10 |
| Lysyl oxidase-like protein 2 | gi:4959425 | 71.10 | 6.32 | 1.05 | | 1.65 | 0.07 | 1.65 | 0.00 |
| Beta-galactosidase binding lectin | gi:12804557 | 14.72 | 5.33 | | C | 1.36 | 0.18 | 1.27 | 0.12 |
| Nm23 protein | gi:35068 | 20.41 | 7.06 | 1.04 | | 2.50 | 0.00 | | |
| X-linked brain specific factor | gi:21322252 | 99.19 | 6.00 | | | | | | |
| BCL6 corepressor | gi:21040336 | 78.85 | 8.28 | | | | | | |
| Dickkopf-1* | gi:6049604 | 28.67 | 8.80 | | | | | | |
| Growth arrest-specific 6 | gi:7512417 | 43.14 | 5.26 | 1.14 | | 2.08 | 0.12 | 1.72 | 0.11 |
| Collagen type XI alpha-1 | gi:6165881 | 176.65 | 5.24 | 1.01 | | 2.50 | 0.00 | 2.50 | 0.00 |
| Proteoglycan PG-M (V3) | gi:1008913 | 74.25 | 7.43 | 1.34 | C | 2.20 | 0.00 | 2.50 | 0.00 |
| Galectin-1 | gi:12804557 | 14.72 | 5.33 | 1.05 | C | 1.36 | 0.18 | 1.27 | 0.12 |
| Connective tissue growth factor | gi:4503123 | 39.07 | 8.36 | 1.15 | | 1.87 | 0.54 | 1.66 | 0.14 |
| CD83 antigen, activated lympohocytes | gi:4757946 | 23.04 | 8.45 | 1.11 | | 1.94 | 0.46 | 1.27 | 0.00 |
| KIAA0548 | gi:3043620 | 50.08 | 7.61 | 1.17 | C | 2.28 | 0.04 | 2.50 | 0.00 |
| Beta-2 microglobulin | gi:179318 | 13.73 | 6.06 | 1.12 | C | 1.25 | 0.01 | 1.46 | 0.19 |
| Myeloid leukemia-inhibitory factor | gi:187141 | 21.34 | 9.37 | 1.08 | C | 2.44 | 0.00 | 2.36 | 0.00 |
| Macrophage migration inhibitory factor | gi:312334 | 124.76 | 7.73 | 1.07 | C | 1.53 | 0.04 | 1.43 | 0.14 |
| 2-phosphopyruvate-hydratase-enolase | gi:119339 | 47.17 | 7.01 | 1.06 | C | 1.28 | 0.06 | 1.27 | 0.00 |
| Saposin precursor | gi:134218 | 58.11 | 5.06 | 1.18 | C | 1.43 | 0.03 | 1.39 | 0.00 |
| Autotaxin-t | gi:1160616 | 99.02 | 7.14 | 1.14 | | 1.43 | 0.05 | 1.26 | 0.00 |
| Epididymal secretory E1 precursor | gi:48429027 | 16.57 | 7.57 | | | 1.71 | 0.28 | 2.04 | 0.10 |
| u-type plasminogen activator | gi:137112 | 48.53 | 87.80 | 1.09 | | 1.58 | 0.11 | 1.40 | 0.11 |
| PAI-1* | gi:10835159 | 45.59 | 6.68 | | | | | | |
| TIMP-3 | gi:490094 | 23.17 | 8.46 | 1.35 | | 2.01 | 0.16 | 1.55 | 0.17 |
| Glioma pathogenesis-related protein | gi:5803151 | 30.34 | 8.80 | 1.32 | | 2.50 | 0.00 | 2.50 | 0.00 |
| Palmitoyl-hydrolase precursor | gi:2135879 | 34.18 | 6.07 | 1.10 | | 2.75 | 0.00 | 2.50 | 0.00 |
| Mono-ADP-ribosyltransferase | gi:47087626 | 5.56 | 4.34 | 1.07 | | 1.49 | 0.04 | 1.43 | 0.00 |
| Mitogen-activated protein kinase | gi:66932916 | 41.39 | 6.50 | 1.18 | | 4.02 | 0.67 | 2.96 | 0.28 |
| alpha2-HS glycoprotein/Fetuin-A | gi:7106502 | 39.32 | 5.43 | | | 1.47 | 0.10 | 1.55 | 0.05 |
| Cytosolic thyroid hormone-bp | gi:338827 | 58.00 | 7.95 | 1.22 | | 2.50 | 0.00 | | |
| NF1 protein isoform | gi:219940 | 62.30 | 8.04 | 1.09 | | 2.82 | 0.00 | 2.58 | 0.00 |
| Importin-7 | gi:5453998 | 119.52 | 4.70 | 1.09 | | 1.42 | 0.05 | 1.37 | 0.26 |
| Osteopontin* | gi:189405 | 33.84 | 4.40 | | | | | | |
| Beta 5-tubulin | gi:21104420 | 49.67 | 4.78 | 1.05 | C | 1.55 | 0.25 | 1.59 | 0.09 |
| alpha-catenin | gi:433411 | 100.07 | 5.95 | 1.08 | C | 4.02 | 0.00 | 3.55 | 0.31 |
| BDNP | gi:987872 | 27.76 | 8.77 | 1.01 | C | 2.02 | 0.16 | 1.57 | 0.00 |
| HP1Hs-gamma | gi:1773227 | 19.72 | 5.03 | | | 1.77 | 0.24 | 1.70 | 0.08 |
| KIAA0828 | gi:24308043 | 66.72 | 7.13 | 1.12 | | 1.41 | 0.00 | 1.54 | 0.00 |
| Unnamed protein product | gi:31873592 | 33.25 | 6.51 | | | 1.89 | 0.22 | 1.65 | 0.01 |

| | | | Post-translational |
|---|---|---|---|
| Protein name | Functional category | Mode of Secretion | requirement |
| Galectin-3 | Adhesion and matrix | Vesicle-mediated; Ectocytosis | Glycosylation/ |
| | interactions | | phosphorylation |
| Lysyl oxidase-like protein 2 | Adhesion and matrix | Classical | Glycosylation |
| | interactions | | |
| Beta-galactosidase binding lectin | Cell proliferation | Vesicle-mediated; Ectocytosis | Glycosylation |
| | regulation | | |
| Nm23 protein | Cell proliferation/ | Unknown non-classical pathway | Phosphorylation |
| | differentiation | | |
| X-linked brain specific factor | Cell proliferation/ | Unknown non-classical pathway | Phosphorylation |
| | differentiation | | |
| BCL6 corepressor | DNA binding | Vesicle-mediated; Ectocytosis | Glycosylation |
| Dickkopf-1* | ECM component/signaling | Unknown non-classical pathway | Phosphorylation |
| Growth arrest-specific 6 | ECM component | Classical | Glycosylation |
| Collagen type XI alpha-1 | ECM component | Classical | Glycosylation |
| Proteoglycan PG-M (V3) | ECM component | Non-classical; receptor mediated | Glycosylation |
| Galectin-1 | Cell proliferation | Vesicle-mediated; Ectocytosis | Glycosylation/ |
| | regulation | | phosphorylation |
| Connective tissue growth factor | Cell proliferation/ | Classical | Glycosylation |
| | differentiation | | |
| CD83 antigen, activated lympohocytes | Immunity and defense | Non-classical; receptor mediated | Glycosylation |
| KIAA0548 | Immunity and defense | Unknown non-classical pathway | Glycosylation/ |
| | | | phosphorylation |
| Beta-2 microglobulin | Immunity and defense | Classical | Glycosylation |
| Myeloid leukemia-inhibitory factor | Immunity and defense | Vesicle-mediated; Ectocytosis | Glycosylation |
| Macrophage migration inhibitory factor | Immunity and defense | Vesicle-mediated; Ectocytosis | Glycosylation |
| 2-phosphopyruvate-hydratase-enolase | Metabolic enzyme | Vesicle-mediated; Ectocytosis | Unknown |
| Saposin precursor | Metabolism | Unknown non-classical pathway | Glycosylation |
| Autotaxin-t | Metabolism | Non-classical; receptor mediated | Glycosylation |
| Epididymal secretory E1 precursor | Potease | Ectocytosis | Phosphorylation |
| u-type plasminogen activator | Protease | Classical | Glycosylation |
| PAI-1* | Serine-type protease | Vesicle mediated; Exocytosis | Glycosylation |
| TIMP-3 | Protease inhibitor | Classical | Glycosylation/ |
| | | | phosphorylation |
| Glioma pathogenesis-related protein | Protease inhibitor | Classical | Glycosylation |
| Palmitoyl-hydrolase precursor | Protein synthesis | Classical | Glycosylation |
| Mono-ADP-ribosyltransferase | Protein synthesis | Unknown non-classical pathway | Unknown |
| Mitogen-activated protein kinase | Signalling | Unknown non-classical pathway | Unknown |
| alpha2-HS glycoprotein/Fetuin-A | Signalling | Classical | Glycosylation |
| Cytosolic thyroid hormone-bp | Signalling | Unknown non-classical pathway | Phosphorylation |
| NF1 protein isoform | Signalling | Unknown non-classical pathway | Glycosylation/ |
| | | | phosphorylation |
| Importin-7 | Signalling | Unknown non-classical pathway | Unknown |
| Osteopontin* | Structure and support | Vesicle-mediated; Exocytosis | Glycosylation/ |
| | | | phosphorylation |
| Beta 5-tubulin | Structure and motility | Classical | Phosphorylation |
| alpha-catenin | Structure and motility | Vesicle-mediated; Exocytosis | Glycosylation/ |
| | | | phosphorylation |
| BDNP | Survival | Classical | Glycosylation |
| HP1Hs-gamma | Unknown | Unknown non-classical pathway | Unknown |
| KIAA0828 | Unknown | Unknown non-classical pathway | Glycosylation |
| Unnamed protein product | Unknown | Undetermined | Unknown |

TABLE 2
2024 WT11

| Protein name | Acces. # | MW | pI | mRNA | 2-DE | H:L | st. Dev | H:L | st. Dev |
|---|---|---|---|---|---|---|---|---|---|
| ADAM-10* | gi:4557251 | 84.14 | 8.04 | | | | | | |
| PEDF* | gi:189778 | 46.33 | 5.84 | | C | 0.38 | 0.00 | 0.56 | 0.00 |
| CYR61 protein | gi:12584866 | 41.99 | 8.64 | 1.13 | C | 0.69 | 0.10 | 0.65 | 0.00 |
| VEGF* | gi:181971 | 22.31 | 7.88 | | | | | | |
| Transforming growth factor beta* | gi:339558 | 12.79 | 8.59 | 1.18 | C | | | 0.75 | 0.00 |
| FGF-4 | gi:4503701 | 22.05 | 9.73 | | | | | | |
| RTVP-1* | gi:27735198 | 30.37 | 8.80 | | | | | | |
| TPM4-ALK fusion oncoprotein | gi:10441386 | 27.53 | 4.77 | | | | | | |
| Granulin | gi:183613 | 63.57 | 6.50 | 1.18 | | 0.86 | 0.21 | 0.80 | 0.07 |
| Interleukin 8 | gi:33959 | 10.90 | 9.10 | 1.16 | C | 0.63 | 0.02 | 0.62 | 0.00 |
| Attractin | gi:3676347 | 175.00 | 4.71 | 1.16 | C | 0.39 | 0.16 | 0.27 | 0.09 |
| ANP32A protein | gi:76825059 | 14.79 | 5.27 | 1.21 | | 0.44 | 0.33 | 0.78 | 0.01 |
| Triosephosphate | gi:37247 | 26.67 | 6.45 | 1.04 | | 0.79 | 0.00 | 0.76 | 0.02 |
| Aldolase A | gi:34577112 | 39.42 | 8.30 | 1.37 | | 0.18 | 0.18 | 0.75 | 0.05 |
| MMP-2* | gi:11342666 | 73.88 | 5.26 | | | | | | |
| Helicase-MOI | gi:5019620 | 218.81 | 5.47 | | | 0.23 | 0.00 | 0.25 | 0.00 |
| SPARC/Osteonectin* | gi:4507171 | 34.63 | 4.73 | 1.08 | C | 0.79 | 0.03 | 0.78 | 0.06 |
| A Chain A, Bm-40 | gi:2624793 | 27.01 | 5.53 | 1.12 | C | 0.76 | 0.00 | 0.73 | 0.00 |
| Insulin-like growth factor bp6 | gi:183894 | 25.19 | 8.15 | 1.05 | C | 0.78 | 0.05 | 0.74 | 0.08 |
| Metallothionein II D | gi:223529 | 6.04 | 8.23 | | | | | | |
| Transgelin 2 | gi:4507357 | 22.39 | 8.41 | | | | | | |

| | | | Post-translational |
|---|---|---|---|
| Protein name | Functional category | Mode of Secretion | requirement |
| ADAM-10* | Adhesion | Classical | Glycosylation |
| PEDF* | Angiogenic | Classical | Glycosylation |
| CYR61 protein | Angiogenic | Classical | Unknown |
| VEGF* | Angiogenic | Classical | Glycosylation |
| Transforming growth factor beta* | Cell proliferation/ | Classical | Glycosylation |
| | angiogenic | | |
| FGF-4 | Cell growth/prolifer- | Classical | Glycosylation |
| | ation/angiogenic | | |
| RTVP-1* | Cell proliferation/ | Classical | Glycosylation/ |
| | differentiation | | phosphorylation |
| TPM4-ALK fusion oncoprotein | Cell proliferation | Non-classical; receptor mediated | Glycosylation |
| Granulin | Cell proliferation | Unknown non-classical method | Glycosylation |
| Interleukin 8 | Immunity and defense/ | Classical | Unknown |
| | angiogenic | | |
| Attractin | Immunity and defense | Unknown non-classical pathway | Unknown |
| ANP32A protein | Immunity and defense | Undetermined | Unknown |
| Triosephosphate | Metabolism | Undetermined | Glycosylation |
| Aldolase A | Metabolism | Undetermined | Unknown |
| MMP-2* | Protease | Classical | Glycosylation/ |
| | | | phosphorylation |
| Helicase-MOI | RNA modification | Classical | Glycosylation/ |
| | | | phosphorylation |
| SPARC/Osteonectin* | Structure and motility | Classical | Glycosylation |
| A Chain A, Bm-40 | Structure and motility | Classical | None |
| Insulin-like growth factor bp6 | Survival | Unknown non-classical method | Unknown |
| Metallothionein II D | Unknown | Classical | Glycosylation |
| Transgelin 2 | Unknown | Unknown non-classical method | None |

| | | | | | | | st. | | st. | |
|---|---|---|---|---|---|---|---|---|---|---|
| Protein name | Acces. # | MW | pI | mRNA | 2-DE | H:L | Dev | H:L | Dev | Functional category |
| Thrombospondin-1* | gi:40317626 | 129.38 | 4.71 | 1.13 | C | 1.05 | 0.04 | 1.09 | 0.10 | Anti-angiogenic |
| Endothelin 1 | gi:556203 | 24.43 | 9.52 | 1.06 | C | 1.01 | 0.00 | 0.90 | 0.11 | Angiogenic |
| MAC25 | gi:307151 | 28.75 | 8.40 | 1.15 | | 1.03 | 0.01 | 1.02 | 0.00 | Cell proliferation regulation |
| GDNF family receptor alpha 1 isoform | gi:20141405 | 51.46 | 8.30 | 1.05 | | 1.63 | 0.00 | | | Cell differentiation |
| Neurotrophin | gi:4505469 | 29.35 | 9.34 | | | | | | 0. | Differentiation and survival |
| Stage-specific S antigen homolog | gi:51466832 | 68.80 | 11.82 | | C | 1.09 | 0.06 | 1.12 | 0.03 | DNA-binding protein |
| Hyp. Zinc finger protein KIAA0296 | gi:40788207 | 201.84 | 7.05 | 1.18 | C | 1.08 | 0.02 | 0.91 | 0.03 | DNA-binding protein |
| Ah receptor-interacting protein | gi:6226814 | 37.66 | 6.04 | 1.19 | U | 1.46 | 0.34 | 1.43 | 0.07 | DNA binding protein |
| Fibulin 1A | gi:19743813 | 138.97 | 5.41 | | | | | | | ECM component; cell signalling |
| Prion protein | gi:220016 | 26.82 | 9.04 | 1.23 | | 0.14 | 0.20 | 0.96 | 0.00 | ECM protein |
| Procollagen C-endopeptidase enhancer | gi:4505643 | 47.95 | 7.41 | 1.11 | | 1.18 | 0.02 | 1.45 | 0.08 | Enzyme |
| Phosphodiesterase I alpha | gi:662290 | 99.04 | 7.49 | 1.25 | | 1.22 | 0.00 | 1.09 | 0.08 | Enzyme |
| Follistatin-like 1 | gi:2498390 | 34.99 | 5.39 | 1.06 | C | 1.11 | 0.02 | 1.03 | 0.03 | Immunity and defense |
| Immunoglobulin, heavy chain variable | gi:553428 | 16.16 | 9.52 | 1.03 | U | 1.45 | 0.00 | 1.45 | 0.09 | Immunity and defense |
| MAC-2 binding protein precursor | gi:41150551 | 52.47 | 8.91 | | C | 0.98 | 0.16 | 1.02 | 0.06 | Immunity and defense |
| T cell receptor alpha chain | gi:416065 | 11.39 | 6.10 | 1.04 | C | 0.86 | 0.00 | 1.10 | 0.15 | Immunity and defense |
| Immunoglobulin kappa chain variable | gi:185974 | 12.50 | 5.14 | 1.07 | C | 0.94 | 0.00 | 0.94 | 0.05 | Immunity and defense |
| Glycosylase I | gi:62821794 | 66.22 | 8.31 | | | 1.06 | 0.02 | 1.04 | 0.04 | Metabolism |
| T-cell receptor delta chain | gi:540457 | 12.98 | 5.50 | 1.03 | U | 1.00 | 0.00 | 1.00 | 0.00 | Metabolic glycolytic enzyme |
| Isomerase, triosephosphate | gi:223374 | 26.63 | 7.09 | 1.04 | C | 1.00 | 0.05 | 1.09 | 0.10 | Immunity and defense |
| HSP 70/71; isoform2 | gi:24234686 | 53.52 | 5.62 | 1.11 | C | 1.02 | 0.10 | 1.04 | 0.00 | Oxidative damage repair enzyme |
| Rnase H | gi:52000844 | 91.95 | 9.21 | | | | | | | RNA degradation |
| Cathepsin B preproprotein | gi:22538437 | 37.85 | 5.88 | 1.21 | C | 0.98 | 0.05 | 1.00 | 0.06 | Protease |
| heterogeneous nuclear ribonucleoprotein | gi:14165439 | 51.03 | 5.19 | 1.08 | U | 0.75 | 0.14 | 0.78 | 0.09 | Protein synthesis |
| RNA binding protein regulatory subunit | gi:14198257 | 19.89 | 6.33 | 1.08 | | 1.00 | 0.00 | | | Protein synthesis |
| Ribosomal protein S12 | gi:14277700 | 14.51 | 6.81 | 1.18 | C | 1.37 | 0.00 | | | Protein synthesis |
| CAMKII | gi:25952118 | 54.09 | 6.61 | | | | | | | Kinase inhibitor |
| Calmodulin related protein-A11 | gi:47458820 | 83.13 | 6.70 | | U | 1.01 | 0.06 | 1.12 | 0.03 | Protein kinase |
| Fibrillin-2 pre | gi:66346695 | 314.77 | 4.73 | 1.06 | C | 0.83 | 0.05 | 0.93 | 0.07 | Structure and motility |
| 14-3-3 protein tau | gi:112690 | 27.76 | 4.68 | 1.11 | U | 0.80 | 0.12 | 0.87 | 0.00 | Structure and motility |
| Cofilin, non-muscle isoform | gi:5031635 | 18.52 | 8.22 | 1.07 | C | 0.93 | 0.02 | 0.97 | 0.05 | Structure and motility |
| Filamin | gi:8100574 | 278.20 | 5.49 | 1.15 | U | 0.00 | 0.00 | 2.03 | 0.16 | Structure and motility |
| Vimentin | gi:62414289 | 53.65 | 5.06 | 1.06 | | 0.87 | 0.04 | 0.96 | 0.00 | Structure and motility |
| Myosin light chain 3 | gi:4557777 | 21.93 | 5.03 | 1.06 | C | 1.06 | 0.08 | 1.13 | 0.01 | Structure and motility |
| Stanniocalcin 2 precursor | gi:4507267 | 33.25 | 6.93 | 1.21 | C | 1.07 | 0.05 | 1.10 | 0.11 | Signalling |
| Tyrosine 3-monooxygenase | gi:136574 | 58.52 | 5.90 | 1.16 | | 1.04 | 0.13 | 1.12 | 0.04 | Signalling |
| Peptidylprolyl isomerase A | gi:10863927 | 18.01 | 7.68 | 1.02 | | 1.24 | 0.02 | 1.21 | 0.00 | Signalling |
| YWHAZ protein | gi:68085909] | 27.75 | 4.73 | 1.18 | | 1.31 | 0.21 | 1.24 | 0.00 | Signalling |
| Insulin-like growth Factor bp5 precursor | gi:184820 | 30.57 | 8.58 | 1.25 | U | 1.92 | 0.10 | 1.30 | 0.09 | Survival; immunity and defense |
| Thioredoxin/NKEF-B | gi:50592994 | 11.74 | 4.82 | 1.08 | U | 1.64 | 0.37 | 1.63 | 0.00 | Survival/immunity |
| Amyloid A4 protein | gi:28721 | 84.82 | 4.71 | 1.28 | | 1.12 | 0.07 | 1.06 | 0.08 | Transport |
| Apolipoprotein-E | gi:671882 | 11.84 | 6.57 | | | | | | | Transport |
| Human serum albumin | gi:178344 | 69.37 | 5.92 | | | | | | | Transport |
| GDI-alpha | gi:4757768 | 23.21 | 5.03 | | | | | | | Vesicle-mediated transport |
| CDw44 antigen precursor | gi:180197 | 39.56 | 5.13 | 1.22 | | 0.85 | 0.16 | 0.82 | 0.05 | Unknown |
| Unnamed protein product; phosphatase | gi:19701027 | 77.58 | 5.05 | | U | 1.15 | 0.06 | 1.31 | 0.02 | Unknown |
| Similar to HSPC280 | gi:6841210 | 15.80 | 7.91 | | U | 0.90 | 0.09 | 0.97 | 0.00 | Unknown |
| Hypothetical FGF-like protein | gi:4557679 | 133.10 | 5.71 | | | 1.15 | 0.04 | 1.33 | 0.17 | Unknown |
| KIAA0012 | gi:40789057 | 90.55 | 6.64 | | | 1.31 | 0.21 | 1.17 | 0.00 | Unknown |
| MGC:71022 | gi:38303909 | 10.83 | 4.69 | | | 1.23 | 0.00 | 0.99 | 0.00 | Unknown |
| Unnamed protein product; zinc finger | gi:21751981 | 82.65 | 6.78 | | | 1.69 | 0.08 | 0.97 | 0.00 | Unknown |

TABLE 4

| Protein name | Acces. # | Th. MW | Th. pI | Obs. MW | Obs. pI | Potential post-translational modification |
|---|---|---|---|---|---|---|
| Galectin-3 | gi:4504983 | 26.19 | 8.58 | 25-35 | 7.5-8.7 | Phosphorylation and/or glycosylation |
| Galectin-1 | gi:12804557 | 14.72 | 5.33 | 17-33 | 5.0-5.5 | Phosphorylation and/or glycosylation |
| Beta-2 microglobulin | gi:179318 | 13.73 | 6.06 | 15 | 5.0-5.5 | Phosphorylation |
| Proteoglycan PG-M (V3) | gi:1008913 | 74.25 | 7.43 | 1.34 | 7.5-7.75 | Dephosphorylation and/or Deglycosylation |
| Cytosolic thyroid hormone-bp | gi:338827 | 58.00 | 7.95 | 57-62 | 6.4-6.5 | Dephosphorylation and/or Deglycosylation |
| ADAM-10 | gi:4557251 | 84.14 | 8.04 | 70 | 8.7-9.0 | Dephosphorylation or proteolysis |
| MMP-2 | gi:11342666 | 73.88 | 5.26 | 70-75 | 4.1 | Not determined |
| Thrombospondin-1 | gi:40317626 | 129.38 | 4.71 | 125 | 4.5-4.7 | Phosphorylation |
| 14-3-3 protein tau | gi:112690 | 27.76 | 4.68 | 13 | 4.0-5.0 | Dephosphorylation and/or proteolysis |

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not be limited to the specific embodiments disclosed and that modifications and other embodiments are intended to be included within the scope of the appended claims. Although specific terms are employed herein, they are used in a generic and descriptive sense only and not for purpose of limitation. Further, it must be noted that as used in this specification and the appended embodiments, the singular forms “a,” “an,” and “the” include plural references unless the context clearly dictates otherwise.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

### REFERENCES

Aikawa, S., Y. Hatta, M. Tanaka, Y. Kaneita, K. Yasukawa, U. Sawada, T. Horie, I. Tsuboi and S. Aizawa (2003). Requirement of soluble factors produced by bone marrow stromal cells on the growth of novel established human myeloma cell line. Int J Oncol 22, 631-7.
Albertoni, M., D. M. Daub, K. C. Arden, C. S. Viars, C. Powell, et al. (1998). “Genetic instability leads to loss of both p53 alleles in a human glioblastoma.” Oncogene 16(3): 321-6.
Albertoni, M., P. H. Shaw, M. Nozaki, S. Godard, M. Tenan, et al. (2002). “Anoxia induces macrophage inhibitory cytokine-1 (MIC-1) in glioblastoma cells independently of p53 and HIF-1.” Oncogene 21(27): 4212-9.
Amzallag, N., B. J. Passer, D. Allanic, E. Segura, C. Thery, et al. (2004). “TSAP6 facilitates the secretion of translationally controlled tumor protein/histamine-releasing factor via a nonclassical pathway.” J Biol Chem 279(44): 46104-12.
Babic, A. M., M. L. Kireeva, T. V. Kolesnikova and L. F. Lau (1998). “CYR61, a product of a growth factor-inducible immediate early gene, promotes angiogenesis and tumor growth.” Proc Natl Acad Sci USA 95(11): 6355-60.
Bhowmick, N. A. and H. L. Moses (2005). “Tumor-stroma interactions.” Curr Opin Genet Dev 15(1): 97-101.
Bouvet, M., L. M. Ellis, M. Nishizaki, T. Fujiwara, W. Liu, C. D. Bucana, B. Fang, J. J. Lee and J. A. Roth (1998). Adenovirus-mediated wild-type p53 gene transfer down-regulates vascular endothelial growth factor expression and inhibits angiogenesis in human colon cancer. Cancer Res 58, 2288-92.
Braithwaite, A. W., J. A. Royds and P. Jackson (2005). The p53 story: layers of complexity. Carcinogenesis 26, 1161-9.
Brat, D. J., A. C. Bellail and E. G. Van Meir (2005). “The role of interleukin-8 and its receptors in gliomagenesis and tumoral angiogenesis.” Neuro-oncol 7(2): 122-33.
Bueter, M., M. Gasser, T. Lebedeva, G. Benichou and A. M. Waaga-Gasser (2006). “Influence of p53 on anti-tumor immunity (review).” Int J Oncol 28(2): 519-25.
Chiarugi, V., L. Magnelli and O. Gallo (1998). “Cox-2, iNOS and p53 as play-makers of tumor angiogenesis (review).” Int J Mol Med 2(6): 715-9.
Chipuk, J. E., T. Kuwana, L. Bouchier-Hayes, N. M. Droin, D. D. Newmeyer, M. Schuler and D. R. Green (2004). Direct activation of Bax by p53 mediates mitochondrial membrane permeabilization and apoptosis. Science 303, 1010-4.
Da Costa, L. T., J. Jen, T. C. He, T. A. Chan, K. W. Kinzler and B. Vogelstein (1996). Converting cancer genes into killer genes. Proc Natl Acad Sci USA 93, 4192-6. Dameron K M, Volpert O V, Tainsky M A, Bouck N., Control of angiogenesis in fibroblasts by p53 regulation of thrombospondin-1. Science. 1994 Sep. 9; 265(5178):1582-4.
Debray, C., P. Vereecken, N. Belot, P. Teillard, J. P. Brion, M. Pandolfo and R. Pochet (2004). Multifaceted role of galectin-3 on human glioblastoma cell motility. Biochem Biophys Res Commun 325, 1393-8.
Desbaillets, I., A. C. Diserens, N. Tribolet, M. F. Hamou and E. G. Van Meir (1997). “Upregulation of interleukin 8 by oxygen-deprived cells in glioblastoma suggests a role in leukocyte activation, chemotaxis, and angiogenesis.” J Exp Med 186(8): 1201-12.
Dumic, J., S. Dabelic and M. Flogel (2006). Galectin-3: An open-ended story. Biochim Biophys Acta.
Erster, S. and U. M. Moll (2005). Stress-induced p53 runs a transcription-independent death program. Biochem Biophys Res Commun 331, 843-50.
Fang, B. and J. A. Roth (2003). Tumor-suppressing gene therapy. Cancer Biol Ther 2, S115-21.
Fukumori, T., Y. Takenaka, T. Yoshii, H. R. Kim, V. Hogan, H. Inohara, S. Kagawa and A. Raz (2003). CD29 and CD7 mediate galectin-3-induced type II T-cell apoptosis. Cancer Res 63, 8302-11.
Framson, P. E. and E. H. Sage (2004). “SPARC and tumor growth: where the seed meets the soil?” J Cell Biochem 92(4): 679-90.
Fulci, G. and E. G. Van Meir (1999). “p53 and the CNS: tumors and developmental abnormalities.” Mol Neurobiol 19(1): 61-77.
Fulci, G., N. Ishii, D. Maurici, K. M. Gernert, P. Hainaut, B. Kaur and E. G. Van Meir (2002). Initiation of human astrocytoma by clonal evolution of cells with progressive loss of p53 functions in a patient with a 283H TP53 germ-line mutation: evidence for a precursor lesion. Cancer Res 62, 2897-905.
Goldman, D., C. R. Merril and M. H. Ebert (1980). “Two-dimensional gel electrophoresis of cerebrospinal fluid proteins.” Clin Chem 26(9): 1317-22.
Gorg, A., W. Weiss and M. J. Dunn (2004). “Current two-dimensional electrophoresis technology for proteomics.” Proteomics 4(12): 3665-85.
Gygi, S. P., B. Rist, T. J. Griffin, J. Eng and R. Aebersold (2002). “Proteome analysis of low-abundance proteins using multidimensional chromatography and isotope-coded affinity tags.” J Proteome Res 1(1): 47-54.
Hammond, E. M. and A. J. Giaccia (2005). The role of p53 in hypoxia-induced apoptosis. Biochem Biophys Res Commun 331, 718-25.
Hanahan, D. and R. A. Weinberg (2000). “The hallmarks of cancer.” Cell 100(1): 57-70,
Harris, S. L. and A. J. Levine (2005). “The p53 pathway: positive and negative feedback loops.” Oncogene 24(17): 2899-908.
Hao, C., F. Beguinot, G. Condorelli, A. Trencia, E. G. Van Meir, V. W. Yong, I. F. Parney, W. H. Roa and K. C. Petruk (2001). Induction and intracellular regulation of tumor necrosis factor-related apoptosis-inducing ligand (TRAIL) mediated apotosis in human malignant glioma cells. Cancer Res 61, 1162-70.
Hollstein, M., D. Sidransky, B. Vogelstein and C. C. Harris (1991). p53 mutations in human cancers. Science 253, 49-53.
Ishii, N., D. Maier, A. Merlo, M. Tada, Y. Sawamura, A. C, Diserens and E. G. Van Meir (1999). Frequent co-alterations of TP53, p16/CDKN2A, p14ARF, PTEN tumor suppressor genes in human glioma cell lines. Brain Pathol 9, 469-79.
Ishii, N., M. Tada, M. F. Hamou, R. C. Janzer, K. Meagher-Villemure, O. D. Wiestler, N. Tribolet and E. G. Van Meir (1999). Cells with TP53 mutations in low grade astrocytic tumors evolve clonally to malignancy and are an unfavorable prognostic factor. Oncogene 18, 5870-8.
Kamemura, K. and G. W. Hart (2003). “Dynamic interplay between O-glycosylation and O-phosphorylation of nucleocytoplasmic proteins: a new paradigm for metabolic control of signal transduction and transcription.” Prog Nucleic Acid Res Mol Biol 73: 107-36.
Kaur, B., D. J. Brat, N. S. Devi and E. G. Van Meir (2005). Vasculostatin, a proteolytic fragment of brain angiogenesis inhibitor 1, is an antiangiogenic and antitumorigenic factor. Oncogene 24, 3632-42
Khwaja, F W, Duke-Cohan J S, Brat D J, Van Meir E G. Attractin is elevated in the cerebrospinal fluid (CSF) of malignant astrocytoma patients and mediates glioma cell migration. Clinical Cancer Research, In press, 2006.
Khwaja, F W, P. Svoboda, M. Reed, J. Pohl and E. G. Van Meir. (2006). Identification of secreted proteins regulated by wt-p53 expression in glioma cells through proteomic analysis. Oncogene (In press).
Komarova, E. A., L. Diatchenko, O. W. Rokhlin, J. E. Hill, Z. J. Wang, V. I. Krivokrysenko, E. Feinstein and A. V. Gudkov (1998). Stress-induced secretion of growth inhibitors: a novel tumor suppressor function of p53. Oncogene 17, 1089-96.
Krzeslak, A. and A. Lipinska (2004). Galectin-3 as a multifunctional protein. Cell Mol Biol Lett 9, 305-28.
Lain, S. and D. Lane (2003). Improving cancer therapy by non-genotoxic activation of p53. Eur J Cancer 39, 1053-60.
Lee, R., P. Kermani, K. K. Teng and B. L. Hempstead (2001). “Regulation of cell survival by secreted proneurotrophins.” Science 294(5548): 1945-8.
Levine, A. J., J. Momand and C. A. Finlay (1991). The p53 tumour suppressor gene. Nature 351, 453-6.
Little, J. B., E. I. Azzam, S. M. de Toledo and H. Nagasawa (2002). Bystander effects: intercellular transmission of radiation damage signals. Radiat Prot Dosimetry 99, 159-62.
Mazzocca, A., R. Coppari, R. De Franco, J. Y. Cho, T. A. Libermann, et al. (2005). “A secreted form of ADAMS promotes carcinoma invasion through tumor-stromal interactions.” Cancer Res 65(11): 4728-38.
McCormick, F. (2001). Cancer gene therapy: fringe or cutting edge? Nat Rev Cancer 1, 130-41.
Mehul, B. and R. C. Hughes (1997). Plasma membrane targeting, vesicular budding and release of galectin 3 from the cytoplasm of mammalian cells during secretion. J Cell Sci 110 (Pt 10), 1169-78.
Morimoto, I., Y. Sasaki, S. Ishida, K. Imai and T. Tokino (2002). “Identification of the osteopontin gene as a direct target of TP53.” Genes Chromosomes Cancer 33(3): 270-8.
Nakahara, S., N. Oka and A. Raz (2005). On the role of galectin-3 in cancer apoptosis. Apoptosis 10, 267-75.
Nigro, J. M., S. J. Baker, A. C. Preisinger, J. M. Jessup, R. Hostetter, K. Cleary, S. H. Bigner, N. Davidson, S. Baylin, P. Devilee, et al. (1989). Mutations in the p53 gene occur in diverse human tumour types. Nature 342, 705-8.
Oren, M. (2001). The p53 saga: the good, the bad, and the dead. Harvey Leet 97, 57-82.
Passer, B. J., V. Nancy-Portebois, N. Amzallag, S. Prieur, C. Cans, A. Roborel de Climens, G. Fiucci, V. Bouvard, M. Tuynder, L. Susini, et al. (2003). The p53-inducible TSAP6 gene product regulates apoptosis and the cell cycle and interacts with Nix and the Myt1 kinase. Proc Natl Acad Sci USA 100, 2284-9.
Pietras, K., K. Rubin, T. Sjoblom, E. Buchdunger, M. Sjoquist, et al. (2002). “Inhibition of PDGF receptor signaling in tumor stroma enhances antitumor effect of chemotherapy.” Cancer Res 62(19): 5476-84.
Porkka, K. P., N. N. Nupponen, T. L. Tammela, R. L. Vessella and T. Visakorpi (2003). Human pHyde is not a classical tumor suppressor gene in prostate cancer. Int J Cancer 106, 729-35.
Rizk, N. P., M. Y. Chang, C. El Kouri, P. Seth, L. R. Kaiser, S. M. Albelda and K. M. Amin (1999). The evaluation of adenoviral p53-mediated bystander effect in gene therapy of cancer. Cancer Gene Ther 6, 291-301.
Rosenberg, I., B. J. Cherayil, K. J. Isselbacher and S. Pillai (1991). Mac-2-binding glycoproteins. Putative ligands for a cytosolic beta-galactoside lectin. J Biol Chem 266, 18731-6.
Roth, J. A., D. Nguyen, D. D. Lawrence, B. L. Kemp, C. H. Carrasco, D. Z. Ferson, W. K.
Hong, R. Komaki, J. J. Lee, J. C. Nesbitt, et al. (1996). Retrovirus-mediated wild-type p53 gene transfer to tumors of patients with lung cancer. Nat Med 2, 985-91.
Sasaki, T., C. Brakebusch, J. Engel and R. Timpl (1998). Mac-2 binding protein is a cell-adhesive protein of the extracellular matrix which self-assembles into ring-like structures and binds betal integrins, collagens and fibronectin. Embo J 17, 1606-13.
Schuler, M. and D. R. Green (2005). Transcription, apoptosis and p53: catch-22. Trends Genet 21, 182-7.
Seki, M., J. Iwakawa, H. Cheng and P. W. Cheng (2002). p53 and PTEN/MMAC1/TEP1 gene therapy of human prostate PC-3 carcinoma xenograft, using transferrin-facilitated lipofection gene delivery strategy. Hum Gene Ther 13, 761-73.
Sidransky, D., T. Mikkelsen, K. Schwechheimer, M. L. Rosenblum, W. Cavenee and B. Vogelstein (1992). Clonal expansion of p53 mutant cells is associated with brain tumour progression. Nature 355, 846-7.
Song, J. H., D. K. Song, B. Pyrzynska, K. C. Petruk, E. G. Van Meir and C. Hao (2003). TRAIL triggers apoptosis in human malignant glioma cells through extrinsic and intrinsic pathways. Brain Pathol 13, 539-53.
Steele, R. J. and D. P. Lane (2005). “P53 in cancer: a paradigm for modern management of cancer.” Surgeon 3(3): 197-205.
Stuelten, C. H., S. DaCosta Byfield, P. R. Arany, T. S. Karpova, W. G. Stetler-Stevenson, et al. (2005). “Breast cancer cells induce stromal fibroblasts to express MMP-9 via secretion of TNF-alpha and TGF-beta.” J Cell Sci 118(Pt 10): 2143-53.
Sun, Y., J. M. Cheung, J. Martel-Pelletier, J. P. Pelletier, L. Wenger, et al. (2000). “Wild type and mutant p53 differentially regulate the gene expression of human collagenase-3 (hMMP-13).” J Biol Chem 275(15): 11327-32.
Swisher, S. G., J. A. Roth, J. Nemunaitis, D. D. Lawrence, B. L. Kemp, C. H. Carrasco, D. G. Connors, A. K. El-Naggar, F. Fossella, B. S. Glisson, et al. (1999). Adenovirus-mediated p53 gene transfer in advanced non-small-cell lung cancer. J Natl Cancer Inst 91, 763-71.
Taieb, J., N. Chaput, C. Menard, L. Apetoh, E. Ullrich, et al. (2006). “A novel dendritic cell subset involved in tumor immunosurveillance.” Nat Med 12(2): 214-9.
Tan, C., R. G. de Noronha, A. J. Roecker, B. Pyrzynska, F. Khwaja, Z. Zhang, H. Zhang, Q. Teng, A. C. Nicholson, P. Giannakakou, et al. (2005). Identification of a novel small-molecule inhibitor of the hypoxia-inducible factor 1 pathway. Cancer Res 65, 605-12.
Tenan, M., G. Fulci., M. Albertoni, A. C. Diserens, M. F. Hamou, et al. (2000). “Thrombospondin-1 is downregulated by anoxia and suppresses tumorigenicity of human glioblastoma cells.” J Exp Med 191(10): 1789-98.
Tsai, M. S., A. E. Hornby, J. Lakins and R. Lupu (2000). “Expression and function of CYR61, an angiogenic factor, in breast cancer cell lines and tumor biopsies.” Cancer Res 60(20): 5603-7.
Tsuzuki, T., S. Izumoto, T. Ohnishi, S. Hiraga, N. Arita, et al. (1998). “Neural cell adhesion molecule L1 in gliomas: correlation with TGF-beta and p53.” J Clin Pathol 51(1): 13-7.
Van Meir, E. G., P. J. Polverini, V. R. Chazin, H. J. Su Huang, N. de Tribolet, et al. (1994). “Release of an inhibitor of angiogenesis upon induction of wild type p53 expression in glioblastoma cells.” Nat Genet 8(2): 171-6.
Vogelstein, B. and K. W. Kinzler (2004). Cancer genes and the pathways they control. Nat Med 10, 789-99.
Volmer, M. W., K. Stuhler, M. Zapatka, A. Schoneck, S. Klein-Scory, et al. (2005). “Differential proteome analysis of conditioned media to detect Smad4 regulated secreted biomarkers in colon cancer.” Proteomics 5(10): 2587-601.
Vousden, K. H. and C. Prives (2005). P53 and prognosis: new insights and further complexity. Cell 120, 7-10.
Wahl, G. M. and A. M. Carr (2001). The evolution of diverse biological responses to DNA damage: insights from yeast and p53. Nat Cell Biol 3, E277-86.
Wang, J., J. Shou and X. Chen (2000). “Dickkopf-1, an inhibitor of the Wnt signaling pathway, is induced by p53.” Oncogene 19(14): 1843-8.
Xie, D., D. Yin, H, J. Wang, G. T. Liu, R. Elashoff et al. (2004). “Levels of expression of CYR61 and CTGF are prognostic for tumor progression and survival of individuals with gliomas.” Clin Cancer Res 10(6): 2072-81.
Yang, R. Y., D. K. Hsu and F. T. Liu (1996). Expression of galectin-3 modulates T-cell growth and apoptosis. Proc Natl Acad Sci USA 93, 6737-42.
Yu, X., S. L. Harris and A. J. Levine (2006), “The regulation of exosome secretion: a novel function of the p53 protein.” Cancer Res 66(9): 4795-801.
Zigrino, P., S. Loffek and C. Mauch (2005). “Tumor-stroma interactions: their role in the control of tumor cell invasion.” Biochimie 87(3-4): 321-8.
Zhang, X., M. S. Steiner, A, Rinaldy and Y. Lu (2001). Apoptosis induction in prostate cancer cells by a novel gene product, pHyde, involves caspase-3. Oncogene 20, 5982-90.

What is claimed is:

1. A method for inducing apoptosis in a cancer comprising administering to a cancer cell in need thereof an apoptosis inducing amount of galectin, thereby treating the cancer.

2. The method of claim 1, wherein said galectin is Gal-3, Gal-1 or Gal-7.

3. The method of claim 2, wherein said Gal-3 is a recombinant classically secreted Gal-3 (sGal3).

4. The method of claim 3, wherein said sGal-3 is produced through physiological induction of secretion in a non-native mammalian cell type.

5. The method of claim 1, wherein said cancer comprises glioma, breast cancer, lung cancer, prostate cancer, colon cancer, or ovarian cancer.

6. A method for modulating an extracellular secretome comprising controlling expression of galectin or a downstream effector of galectin, thereby modulating the extracellular secretome.

7. The method of claim 6, wherein said extracellular secretome comprises other extracellular proteins regulated by p53.

8. The method of claim 7, wherein said galectin comprises Gal-3, Gal-1, or Gal-7.

9. The method of claim 6, wherein said downstream effector is tumor suppressor activated pathway-6 (TSAP6).

10. A method for determining a diagnosis or a prognosis of a cancer or tumorgenesis state comprising detecting and comparing in a patient extracellular levels of p53-mediated secretomes before and after a treatment.

11. The method of claim 10, wherein said p53-mediated secretome comprises Gal-3, Gal-1, or Gal-7.

12. The method of claim 10, wherein said downstream effector is tumor suppressor activated pathway-6 (TSAP6).

13. A method of inducing paracrine killing effects in a bystander cell comprising activating p53 in a cell in need thereof.

14. The method of claim 13, wherein said activation of p53 mediates bystander tumor suppressive activity by controlling the secretion of a pro-apoptotic factor.

15. The method of claim 13, wherein said pro-apoptotic factor is Gal-3, Gal-1, or Gal-7.

16. A method for identifying a biomarker or a tumor target comprising determining secretion levels of a cell secretome in the presence and absence of p53 expression, comparing the secretion levels of said secretome in normal cells and cancer cells, and identifying said secretome having extracellular secretion levels regulated by p53 in cancer cells, thereby identifying the biomarker or the tumor target.

17. The method of claim 16, wherein said secretome comprises Gal-3, Gal-1 or Gal-7.

18. A method for predicting sensitivity to Gal3 treatment comprising identifying the presence of a target cell receptor for Gal3.

19. A method of screening a biomarker for cancer comprising detecting expression of a receptor component to Gal-3 or other p53-mediated secretome.


SEQUENCE LISTING (10 sequences)

SEQ ID NO: 1 - DNA, 24 bp, Artificial Sequence
  Primer
agaggttcaagcgattctcctgct

SEQ ID NO: 2 - DNA, 24 bp, Artificial Sequence
  Primer
tgctgaaggtgctcttgctctgta

SEQ ID NO: 3 - DNA, 24 bp, Artificial Sequence
  Primer
agattatatcatggtatatgaaag

SEQ ID NO: 4 - DNA, 24 bp, Artificial Sequence
  Primer
agattatatcatggtatatgaaag

SEQ ID NO: 5 - DNA, 20 bp, Artificial Sequence
  Primer
cctgccctcaacaagatgtt

SEQ ID NO: 6 - DNA, 20 bp, Artificial Sequence
  Primer
ggtgaggctcccctttcttg

SEQ ID NO: 7 - DNA, 21 bp, Artificial Sequence
  Primer
ggtacaagacagtgacaggtc

SEQ ID NO: 8 - DNA, 20 bp, Artificial Sequence
  Primer
gttccttgtggagccggagc

SEQ ID NO: 9 - DNA, 26 bp, Artificial Sequence
  Primer
tgaaggtcggagtcaacggatttggt

SEQ ID NO: 10 - DNA, 24 bp, Artificial Sequence
  Primer
catgtgggccatgaggtccaccac